(19) United States
(12) Patent Application Publication
(10) Pub. No.: US 2023/0365616 A1
(43) Pub. Date: Nov. 16, 2023

(54) CHOLESTANE DERIVATIVES, PREPARATIONS CONTAINING THESE DERIVATIVES AND USE THEREOF

(71) Applicant: UNIVERZITA PALACKEHO V OLOMOUCI, Olomouc (CZ)

(72) Inventors: Miroslav KVASNICA, Olomouc (CZ); Gabriel GONZALEZ, Vrbatky (CZ); Veronika GOROVA, Olsany u Prostejova (CZ); Miroslav STRNAD, Olomouc (CZ)

(21) Appl. No.: 18/030,846

(22) PCT Filed: Oct. 22, 2021
(86) PCT No.: PCT/CZ2021/050116

§ 371 Date: Apr. 7, 2023

(30) Foreign Application Priority Data
Oct. 23, 2020 (CZ) PV 2020-579

(51) Int. Cl. C07J 9/00; A61P 25/28; A61K 9/16; A61K 9/10; A61K 47/10; A61K 47/38; A61K 47/08; A61K 47/20; A61K 47/34; A61K 9/48; A61K 9/20

(52) U.S. Cl. CPC C07J 9/00; A61P 25/28; A61K 9/1611; A61K 9/1641; A61K 9/1617; A61K 9/10; A61K 47/10; A61K 47/38; A61K 47/08; A61K 47/20; A61K 47/34; A61K 9/4825; A61K 9/205; A61K 9/2013; A61K 9/2054

(57) ABSTRACT

The invention relates to brassinosteroid-derived cholestane derivatives, their use in the protection of cell damage, injury and cell death and compositions containing these derivatives. New generation of compounds possess also selective antineurodegenerative properties on neuronal cells and tissues and can be particularly used in the treatment and prophylaxis of neurodegenerative disease, particularly in the treatment and prophylaxis of Parkinson's disease.

### FIELD OF ART

The invention relates to new cholestane derivatives derived from plant hormones brassinosteroids, to their use in protection of cell damage and cell toxicity, and compositions containing these derivatives.

### BACKGROUND ART

Parkinson's disease (PD) as the second most common and motor-related neurodegenerative disease with predicted rise in diagnosed cases reaching to 12 million patients by 2040 (Dorsey et al. J. Parkinson's Dis. 2018, 8, S3). PD is characterized by motor symptoms linked with specific degeneration and loss of approximately 30-70% of dopaminergic neurons in substantia nigra pars compacta and their projections to striatum (Rizek et al. CMAJ 2016, 188, 1157). Among many molecular hallmarks of PD includes enhanced oxidative and nitrosative stress (OS & NS), dysfunction of mitochondria, excitotoxicity, ubiquitin/proteasomal system dysfunction (UPS) and neuroinflammation (Dantuma and Bott, Front. Mol. Neurosci 2014, 7; Cookson and Bandmann, Human Mol Gen 2010, 19, R21. Due to the lack of efficient and not only symptomatic treatment of PD, the drug development is focused on agents with efficient curative effect toward PD degenerative processes. One of the resources are natural compounds which tent to have fewer side effects.

Bioactive molecules linked with Parkinson's disease, especially Panaxatriol saponins from Panax notoginseng demonstrated sufficient neuroprotection against 1-methyl-4-phenyl-1,2,3,6-tetrahydropyridine (MPTP)-induced death of PD neurons and behaviour impairment in vivo (Luo et al. J. Ethnopharmacol. 2011, 133, 448). Another example are ginsenosides, a steroid compounds which showed neuroprotective activity in MPTP model of PD. It also increased levels of DA in striatum and substantia nigra by modulation of expression of several genes (Bcl-2, Bcl, Bax, caspase 3 and tyrosine hydroxylase) (Wang et al., Neurochem. Int. 2009, 54, 43). While curcumin is one of the potent spices possessing broad spectrum of antioxidant and neuroprotective activities responsible for anti-PD activities, its synthetic derivative curcumin glucoside was even more effective being able to inhibit aggregation and toxicity of α-synuclein in dose-dependent manner, reduced apoptosis (caspase 3 and 9), oxidative stress and mitochondrial dysfunction in A53T α-synuclein PC12 cells (Liu et al., Pharmacol. Res. 2011, 63, 439). Next source of natural plant-based disease-modifying therapy can be found in Mucuna pruriens, a plant species rich in antioxidants of natural origin such as coenzyme Q-10 (Co-Q10) and L-DOPA. Application of natural form of L-DOPA in the form of Mucuna pruriens seeds demonstrated efficiency in improvement of PD symptoms, but with better pharmacokinetics profile than traditional form of L-DOPA (Bega and Zadikoff, J. Mov. Dis. 2014, 7, 57). Finally, Co-Q10 and creatine have been one of the most studied natural compounds for the treatment of PD. Co-Q10 showed broad neuroprotective activity in paraquat, rotenone,5 1-methyl-4-phenylpyridinium (MPP+) and MPTP models of PD.6,7 It was also found to improve PD symptoms in preclinical study on primates8 and several clinical studies (Shults et al., Arch. Neur. 2002, 59, 1541; Exp. Neurol. 2004, 188, 491).

It is therefore an object of the present invention to provide a new generation of cholestane derivatives which exhibit potent and selective protective properties on neuronal cells and tissues and can be advantageously used in the treatment and prophylaxis of neurodegenerative diseases, preferably Parkinson's disease.

### DISCLOSURE OF THE INVENTION

The object of this invention are cholestane derivatives of the general formula I,

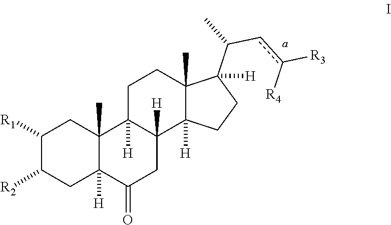

wherein,
a is single or double bond; double bond is independently selected from in either E or Z configuration; R1, R2 is independently selected from the group consisting of hydrogen or hydroxyl group;
R3 is independently selected from the group consisting hydrogen, linear and branched C1-5 alkyl and cycloalkyl;
R4 is independently selected from the group consisting hydrogen, methyl and ethyl;
R3, R4 is independently selected from the bivalent group consisting C2-5 α,ω-alkylene, and wherein the following compounds are excluded from the cholestane derivatives of general formula

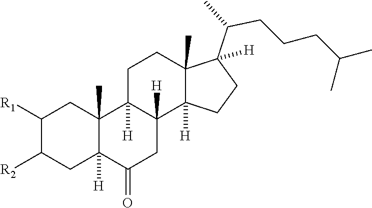

2α-hydroxy-5α-cholestan-6-one, 3α-hydroxy-5α-cholestan-6-one, 2α,3α-dihydroxy-5α-cholestan-6-one, (22E)-2α,3α-dihydroxy-5α-cholest-22-en-6-one, 3α-hydroxy-5α-campestan-6-one, 2α,3α-dihydroxy-5α-campestan-6-one, (22E)-3α-hydroxy-5α-campest-22-en-6-one, (22E)-2α,3α-dihydroxy-α-campest-22-en-6-one, (22E)-2α-hydroxy-5α-ergost-22-en-6-one, (22E)-3α-hydroxy-5α-ergost-22-en-6-one, (22Z)-3α-hydroxy-5α-ergost-22-en-6-one, (22E)-2α,3α-dihydroxy-5α-ergost-22-en-6-one, 3α-hydroxy-24-nor-5α-chol-22-en-6-one, 2α,3α-dihydroxy-24-nor-5α-chol-22-en-6-one, and the pharmaceutically acceptable salts thereof, such as salts with alkali metals, ammonium or amines, or addition salts with acids.

In some embodiments, the compounds of the general formula I bear in position R3 linear C1-5 alkyl which is selected from the group consisting of methyl, ethyl, n-propyl, n-butyl, and n-pentyl.

In some embodiments, the compounds of the general formula I bear in position R3 branched C1-5 alkyl which is selected from the group consisting of group isopropyl, isobutyl, sec-butyl, tert-butyl, 2-methylbut-2-yl, 2,2-dimethylpropyl, 3-methylbut-1-yl, pent-2-yl, pent-3-yl, 3-methylbut-2-yl, and 2-methylbutyl.

In yet some embodiments, the compounds of the general formula I bear in position R3 cycloalkyl group which is selected from the group consisting of cyclopropyl, cyclobutyl, cyclopentyl, cyclohexyl, cyclopropylmethyl, cyclobutylmethyl, cyclopentylmethyl, cyclohexylmethyl, which can be independently at each occurrence substituted by linear C1-5 alkyl.

In yet some embodiments, the compounds of the general formula I bear in position R3 and R4 alkylene group which is selected from the group consisting of 1,2-ethylene, trimethylene, tetramethylene, and pentamethylene.

In yet some embodiments, the compounds of the general formula I bear in position R3 a chiral centre than the absolute configuration is either R or S.

Individual compounds as shown in the examples represent individual preferred embodiments of the present invention. Preferred compounds of the invention are the following compounds of the general formula I derived from the presented structures:

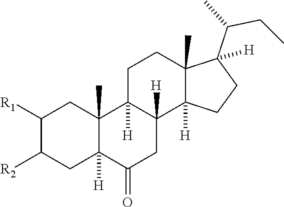

2α-hydroxy-24-nor-5α-cholan-6-one, 3α-hydroxy-24-nor-5α-cholan-6-one, 2α,3α-dihydroxy-24-nor-5α-cholan-6-one;

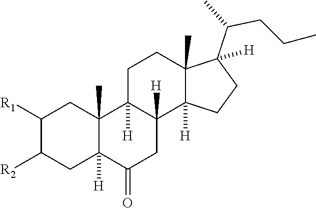

2α-hydroxy-5α-cholan-6-one, 3α-hydroxy-5α-cholan-6-one, 2α,3α-dihydroxy-5α-cholan-6-one;

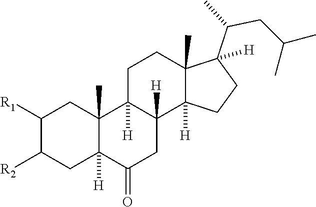

2α-hydroxy-23-methyl-5α-cholan-6-one, 3α-hydroxy-23-methyl-5α-cholan-6-one, 2α,3α-dihydroxy-23-methyl-5α-cholan-6-one;

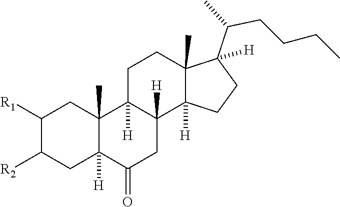

2α-hydroxy-26,27-dinor-5α-cholestan-6-one, 3α-hydroxy-26,27-dinor-5α-cholestan-6-one, 2α,3α-dihydroxy-26,27-dinor-5α-cholestan-6-one;

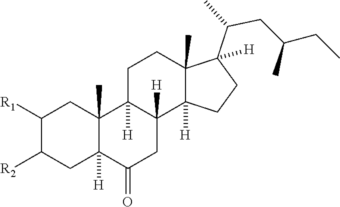

(23R)-2α-hydroxy-23-ethyl-5α-cholan-6-one, (23R)-3α-hydroxy-23-ethyl-5α-cholan-6-one, (23R)-2α,3α-dihydroxy-23-ethyl-5α-cholan-6-one;

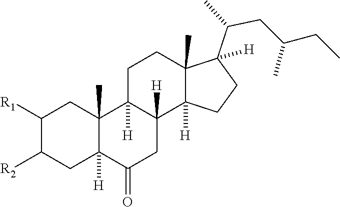

(23S)-2α-hydroxy-23-ethyl-5α-cholan-6-one, (23S)-3α-hydroxy-23-ethyl-5α-cholan-6-one, (23S)-2α,3α-dihydroxy-23-ethyl-5α-cholan-6-one;

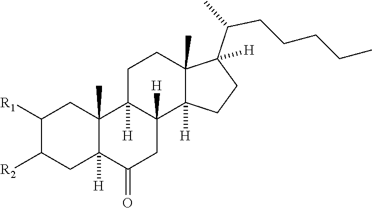

2α-hydroxy-27-nor-5α-cholestan-6-one, 3α-hydroxy-27-nor-5α-cholestan-6-one, 2α,3α-dihydroxy-27-nor-5α-cholestan-6-one;

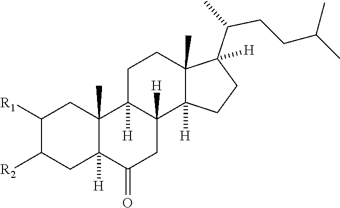

2α-hydroxy-24,24-dimethyl-5α-cholan-6-one, 3α-hydroxy-24,24-dimethyl-5α-cholan-6-one, 2α,3α-dihydroxy-24,24-dimethyl-5α-cholan-6-one;

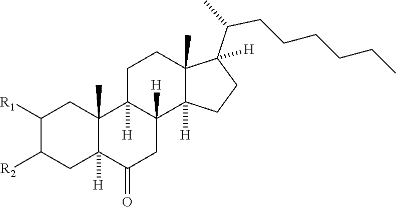

2α-hydroxy-26α-homo-27-nor-5α-cholestan-6-one, 3α-hydroxy-26α-homo-27-nor-5α-cholestan-6-one, 2α,3α-dihydroxy-26α-homo-27-nor-5α-cholestan-6-one;

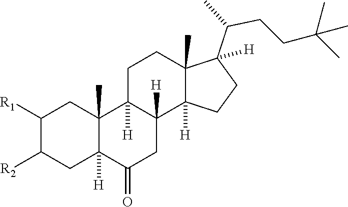

2α-hydroxy-24,24,24-trimethyl-5α-cholan-6-one, 3α-hydroxy-24,24,24-trimethyl-5α-cholan-6-one, 2α,3α-dihydroxy-24,24,24-trimethyl-5α-cholan-6-one;

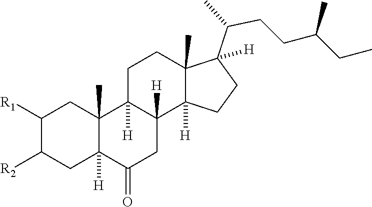

2α-hydroxy-27-nor-5α-campestan-6-one, 3α-hydroxy-27-nor-5α-campestan-6-one, 2α,3α-dihydroxy-27-nor-5α-campestan-6-one;

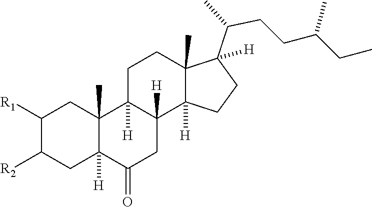

2α-hydroxy-27-nor-5α-ergostan-6-one, 3α-hydroxy-27-nor-5α-ergostan-6-one, 2α,3α-dihydroxy-27-nor-5α-ergostan-6-one;

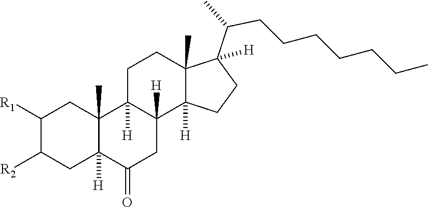

2α-hydroxy-26α,26b-dihomo-27-nor-5α-cholestan-6-one, 3α-hydroxy-26α,26b-dihomo-27-nor-5α-cholestan-6-one, 2α,3α-dihydroxy-26α,26b-dihomo-27-nor-5α-cholestan-6-one;

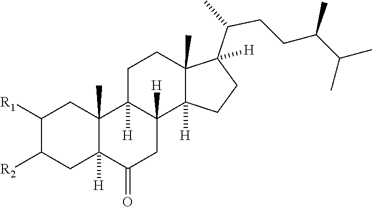

2α-hydroxy-5α-campestan-6-one, 3α-hydroxy-5α-campestan-6-one, 2α,3α-dihydroxy-5α-campestan-6-one;

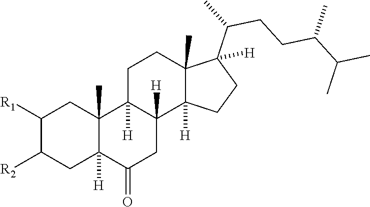

2α-hydroxy-5α-ergostan-6-one, 3α-hydroxy-5α-ergostan-6-one, 2α,3α-dihydroxy-5α-ergostan-6-one;

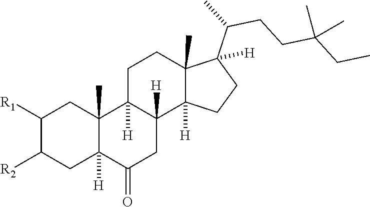

2α-hydroxy-24-ethyl-24,24-dimethyl-5α-cholan-6-one, 3α-hydroxy-24-ethyl-24,24-dimethyl-5α-cholan-6-one, 2α,3α-dihydroxy-24-ethyl-24,24-dimethyl-5α-cholan-6-one;

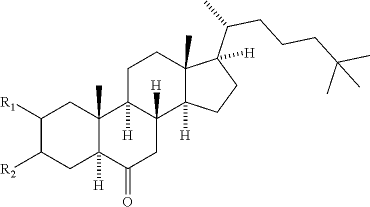

2α-hydroxy-25-methyl-5α-cholestan-6-one,3α-hydroxy-25-methyl-5α-cholestan-6-one, dihydroxy-25-methyl-5α-cholestan-6-one;

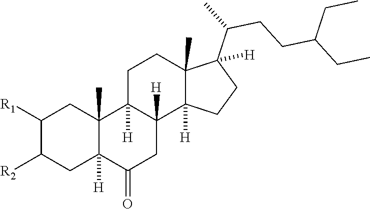

2α-hydroxy-27-nor-5α-stigmastan-6-one, 3α-hydroxy-27-nor-5α-stigmastan-6-one, 2α,3α-dihydroxy-27-nor-5α-stigmastan-6-one;

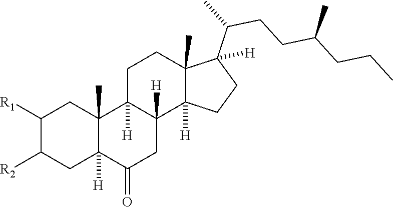

(24S)-2α-hydroxy-26α-homo-27-nor-5α-campestan-6-one, (24S)-3α-hydroxy-26α-homo-27-nor-5α-campestan-6-one, (24S)-2α,3α-dihydroxy-26α-homo-27-nor-5α-campestan-6-one;

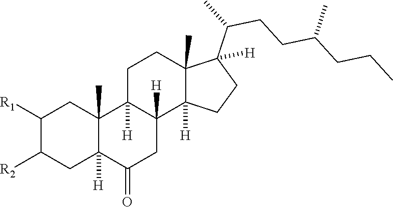

(24R)-2α-hydroxy-26α-homo-27-nor-5α-ergostan-6-one, (24R)-3α-hydroxy-26α-homo-27-nor-5α-ergostan-6-one, (24R)-2α,3α-dihydroxy-26α-homo-27-nor-5α-ergostan-6-one;

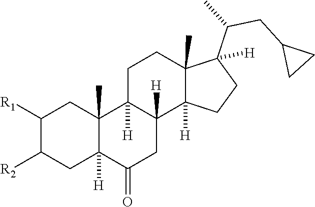

2α-hydroxy-22-cyclopropyl-23,24-dinor-5α-cholan-6-one, 3α-hydroxy-22-cyclopropyl-23,24-dinor-5α-cholan-6-one, 2α,3α-dihydroxy-22-cyclopropyl-23,24-dinor-5α-cholan-6-one;

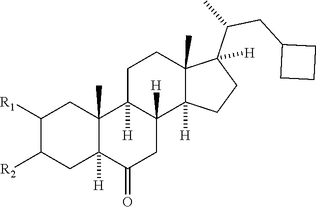

2α-hydroxy-22-cyclobutyl-23,24-dinor-5α-cholan-6-one, 3α-hydroxy-22-cyclobutyl-23,24-dinor-5α-cholan-6-one, 2α,3α-dihydroxy-22-cyclobutyl-23,24-dinor-5α-cholan-6-one;

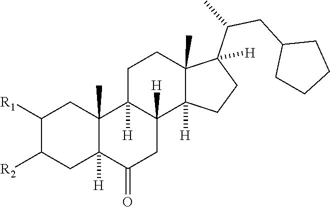

2α-hydroxy-22-cyclopentyl-23,24-dinor-5α-cholan-6-one, 3α-hydroxy-22-cyclopentyl-23,24-dinor-5α-cholan-6-one, 2α,3α-dihydroxy-22-cyclopentyl-23,24-dinor-5α-cholan-6-one;

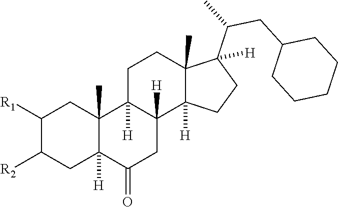

2α-hydroxy-22-cyclohexyl-23,24-dinor-5α-cholan-6-one, 3α-hydroxy-22-cyclohexyl-23,24-dinor-5α-cholan-6-one, 2α,3α-dihydroxy-22-cyclohexyl-23,24-dinor-5α-cholan-6-one;

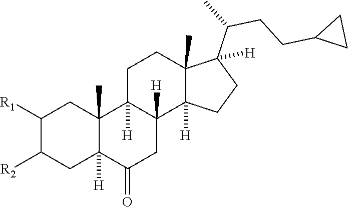

2α-hydroxy-23-cyclopropyl-24-nor-5α-cholan-6-one, 3α-hydroxy-23-cyclopropyl-24-nor-5α-cholan-6-one, 2α,3α-dihydroxy-23-cyclopropyl-24-nor-5α-cholan-6-one;

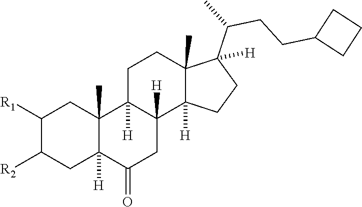

2α-hydroxy-23-cyclobutyl-24-nor-5α-cholan-6-one, 3α-hydroxy-23-cyclobutyl-24-nor-5α-cholan-6-one, 2α,3α-dihydroxy-23-cyclobutyl-24-nor-5α-cholan-6-one;

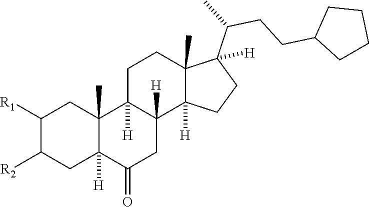

2α-hydroxy-23-cyclopentyl-24-nor-5α-cholan-6-one, 3α-hydroxy-23-cyclopentyl-24-nor-5α-cholan-6-one, 2α,3α-dihydroxy-23-cyclopentyl-24-nor-5α-cholan-6-one;

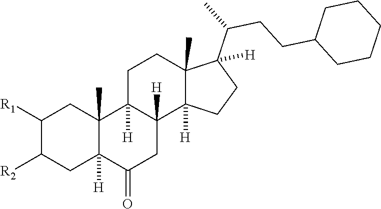

2α-hydroxy-23-cyclohexyl-24-nor-5α-cholan-6-one, 3α-hydroxy-23-cyclohexyl-24-nor-5α-cholan-6-one, 2α,3α-dihydroxy-23-cyclohexyl-24-nor-5α-cholan-6-one.

Generally, the most preferred compounds of the general formula I are: 2α,3α-dihydroxy-24-nor-5α-cholan-6-one, 2α,3α-dihydroxy-5α-cholan-6-one, 2α,3α-dihydroxy-26,27-dinor-5α-cholestan-6-one, 2α,3α-dihydroxy-27-nor-5α-cholestan-6-one, 2α,3α-dihydroxy-5α-cholestan-6-one, 2α,3α-dihydroxy-23-methyl-5α-cholan-6-one, 2α,3α-dihydroxy-23-cyclohexyl-24-nor-5α-cholan-6-one, 2α,3α-dihydroxy-23-cyclopentyl-24-nor-5α-cholan-6-one, 2α,3α-dihydroxy-24,24-dimethyl-5α-cholan-6-one.

The compounds of the present invention have a wide range of biological activities, including activities in increasing viability of neuronal cells, reducing oxidative stress, neuroprotectivity and antiapoptotic activation, which are especially useful in pharmaceutical applications to treat neurodegenerative diseases and correspond to the spectrum of effects required of the agents intended for such treatment.

This invention also provides the compounds of the general formula I for use as antioxidants for inhibiting adverse metabolic processes in mammals and plants either in vivo or in vitro.

The present invention also provides the compounds of the general formula I for use as medicaments.

The invention preferably relates to the compounds of the general formula I for use in the treatment or prophylaxis of neurodegenerative diseases, in particular selected from amyotrophic lateral sclerosis, Parkinson's disease, Alzheimer's disease, Huntington's disease, progressive supranuclear palsy, corticobasal degeneration, frontotemporal dementia, Lewy body dementia, multiple system atrophy, chronic traumatic encephalopathy, spinocerebellar ataxias.

In a preferred embodiment, the invention provides the compounds of the general formula I for use in the treatment and prophylaxis of Parkinson's disease.

The present invention further provides pharmaceutical compositions comprising one or more compounds of the general formula I together with at least one pharmaceutically acceptable carrier.

### Pharmaceutical Compositions

Suitable routes for administration include oral, rectal, topical (including dermal, ocular, buccal and sublingual), vaginal and parenteral (including subcutaneous, intramuscular, intravitreous, intravenous, intradermal, intrathecal and epidural).

The therapeutic compositions generally comprise about 1% to about 95% of the active ingredient. Single-dose forms of administration preferably comprise about 20% to about 90% of the active ingredient and administration forms which are not single-dose preferably comprise about 5% to about 20% of the active ingredient. Unit dose forms are, for example, coated tablets, tablets, ampoules, vials, suppositories or capsules. Other forms of administration are, for example, ointments, creams, pastes, foams, tinctures, lipsticks, drops, sprays, dispersions and the like. Examples are capsules containing from about 0.05 g to about 1.0 g of the active ingredient.

The pharmaceutical and cosmetic compositions of the present invention are prepared in a manner known per se, for example by means of conventional mixing, granulating, coating, dissolving or lyophilizing processes.

Preferably, solutions of the active ingredient, and in addition also suspensions or dispersions, especially isotonic aqueous solutions, dispersions or suspensions, are used, it being possible for these to be prepared before use, for example in the case of lyophilized compositions which comprise the active substance by itself or together with a carrier, for example mannitol. The compositions can be sterilized and/or comprise excipients, for example preservatives, stabilizers, wetting agents and/or emulsifiers, solubilizing agents, salts for regulating the osmotic pressure and/or buffers, and they are prepared in a manner known per se, for example by means of conventional dissolving or lyophilizing processes. The solutions or suspensions mentioned can comprise viscosity-increasing substances, such as sodium carboxymethylcellulose, carboxymethylcellulose, dextran, polyvinylpyrrolidone or gelatin.

Suspensions in oil comprise, as the oily component, vegetable, synthetic or semi-synthetic oils customary for injection purposes. Oils which may be mentioned are, in particular, liquid fatty acid esters which contain, as the acid component, a long-chain fatty acid having 8-22, in particular 12-22, carbon atoms (e.g., lauric acid, tridecylic acid, myristic acid, pentadecylic acid, palmitic acid, margaric acid, stearic acid, acid, arachidonic acid, behenic acid, and the like) or corresponding unsaturated acids (e.g., oleic acid, elaidic acid, euric acid, brasidic acid or linoleic acid). Other additional ingredients known in the art can be included if desired (e.g., antioxidants such as vitamin E, (3-carotene, or 3,5-di-tert-butyl-4-hydroxytoluene, and the like). The alcohol component of these fatty acid esters generally contains no more than about 6 carbon atoms and can be mono- or polyhydric. Mono-, di-, or trihydric alcohols such as methanol, ethanol, propanol, butanol, or pentanol, or isomers thereof, can be used; glycols and glycerols are generally preferred. Fatty acid esters can therefore include, for example, ethyl oleate, isopropyl myristate, isopropyl palmitate, “Labrafil M 2375” (polyoxyethylene glycerol trioleate from Gattefoseé, Paris), “Labrafil M 1944 CS” (unsaturated polyglycolated glycerides prepared by an alcoholysis of apricot kernel oil and made up of glycerides and polyethylene glycol esters; from Gattefoseé, Paris), “Labrasol” (saturated polyglycolated glycerides prepared by an alcoholysis of TCM and made up of glycerides and polyethylene glycol esters; from Gattefoseé, Paris), and/or “Miglyol 812” (triglyceride of saturated fatty acids of chain length C8 to C12 from Hüls AG, Germany), and in particular vegetable oils, such as cottonseed oil, almond oil, olive oil, castor oil, sesame oil, soybean oil and, in particular, groundnut oil as well as mixtures thereof.

The preparation of the compositions intended for human use should, of course, be carried out in the customary and approved manner under sterile conditions, and maintained under appropriate conditions up to and including the time of use.

For example, pharmaceutical compositions for oral use can be obtained by combining the active ingredient with one or more solid carriers, if appropriate granulating the resulting mixture, and, if desired, processing the mixture or granules to tablets or coated tablet cores, if appropriate by addition of additional excipients. Suitable carriers are, in particular, fillers, such as sugars, for example lactose, sucrose, mannitol or sorbitol, cellulose preparations and/or calcium phosphates, for example tricalcium diphosphate, or calcium hydrogen phosphate, and furthermore binders, such as starches, for example maize, wheat, rice or potato starch, methylcellulose, hydroxypropylmethylcellulose, sodium carboxymethylcellulose and/or polyvinylpyrrolidone, and/or, if desired, desintegrators, such as the above mentioned starches, and furthermore carboxymethyl-starch, cross-linked polyvinylpyrrolidone, alginic acid or a salt thereof, such as sodium alginate. Additional excipients are, in particular, flow regulators and lubricants, for example salicylic acid, talc, stearic acid or salts thereof, such as magnesium stearate or calcium stearate, and/or polyethylene glycol, or derivatives thereof.

Coated tablet cores can be provided with suitable coatings which, if appropriate, are resistant to gastric juice, the coatings used being, inter alia, concentrated sugar solutions, which, if appropriate, comprise gum arabic, talc, polyvinylpyrrolidone, polyethylene glycol and/or titanium dioxide, coating solutions in suitable organic solvents or solvent mixtures or, for the preparation of coatings which are resistant to gastric juice, solutions of suitable cellulose preparations, such as acetylcellulose phthalate or hydroxypropylmethylcellulose phthalate. Dyes or pigments can be admixed to the tablets or coated tablet coatings, for example for identification or characterization of different doses of active ingredient. Pharmaceutical compositions, which can be used orally, can also be in the form hard capsules of gelatine and soft, closed capsules of gelatine and a plasticizer, such as glycerol or sorbitol. The hard capsules can contain the active ingredient in the form of granules, mixed for example with fillers, such as maize starch, binders and/or lubricants, such as talc or magnesium stearate, and stabilizers if appropriate. In soft capsules, the active ingredient is preferably dissolved or suspended in suitable liquid excipients, such as greasy oils, paraffin oil or liquid polyethylene glycol's or fatty acid esters of ethylene glycol or propylene glycol, it being likewise possible to add stabilizers and detergents such as, for example, the polyethylene sorbitan fatty acid ester type.

Other oral forms of administration include, for example, syrups prepared in the customary manner, which comprise the active ingredient, for example, in suspended form and in a concentration of about 5% to 20%, preferably about 10% or in a similar concentration which results in a suitable individual dose, for example, when 5 or 10 mL are measured out. Other forms include pulverulent or liquid concentrates for preparing shakes, beverages, and the like. Such concentrates can also be packed in unit dose quantities.

Pharmaceutical compositions, which can be used rectally, are, for example, suppositories that comprise a combination of the active ingredient with a suppository base. Suitable suppository bases are, for example, naturally occurring or synthetic triglycerides, paraffin hydrocarbons, polyethylene glycols or higher alkanols.

Compositions which are suitable for parental administration are aqueous solutions of an active ingredient in water-soluble form, for example of water-soluble salt, or aqueous injection suspensions, which comprise viscosity-increasing substances, for example sodium carboxymethylcellulose, sorbitol and/or dextran, and if appropriate, stabilizers. The active ingredient can also be present here in the form of a lyophilizate, if appropriate together with excipients, and be dissolved before parenteral administration by addition of suitable solvents. Solutions such as are used, for example, for parental administration can also be used as infusion solutions. Preferred preservatives are, for example antioxidants, such as ascorbic acid, or microbicides, such as sorbic or benzoic acid.

Ointments are oil-in-water emulsions, which comprise not more than 70%, but preferably 20-50% of water or aqueous phase. The fatty phase consists, in particular, hydrocarbons, for example vaseline, paraffin oil or hard paraffin's, which preferably comprise suitable hydroxy compounds, such as fatty alcohol's or esters thereof, for example cetyl alcohol or wool wax alcohols, such as wool wax, to improve the water-binding capacity. Emulsifiers are corresponding lipophilic substances, such as sorbitan fatty acid esters (Spans), for example sorbitan oleate and/or sorbitan isostearate. Additives to the aqueous phase are, for example, humectants, such as polyalcohols, for example, glycerol, propylene glycol, sorbitol and/or polyethylene glycol, or preservatives and odoriferous substances.

Fatty ointments are anhydrous and comprise, as the base, in particular, hydrocarbons, for example paraffin, vaseline or paraffin oil, and furthermore naturally occurring or semi-synthetic fats, for example, hydrogenated coconut-fatty acid triglycerides, or, preferably, hydrogenated oils, for example hydrogenated groundnut or castor oil, and furthermore fatty acid partial esters of glycerol, for example glycerol mono- and/or distearate, and for example, the fatty alcohols. They also can contain emulsifiers and/or additives mentioned in connection with the ointments which increase uptake of water.

Creams are oil-in-water emulsions, which comprise more than 50% of water. Oily bases used are, in particular, fatty alcohols, for example, lauryl, cetyl or stearyl alcohols, fatty acids, for example palmitic or stearic acid, liquid to solid waxes, for example isopropyl myristate, wool wax or beeswax, and/or hydrocarbons, for example vaseline (petrolatum) or paraffin oil. Emulsifiers are surface-active substances with predominantly hydrophilic properties, such as corresponding non-ionic emulsifiers, for example fatty acid esters of polyalcohols or ethyleneoxy adducts thereof, such as polyglyceric acid fatty acid esters or polyethylene sorbitan fatty esters (Tweens), and furthermore polyoxyethylene fatty alcohol ethers or polyoxyethylene fatty acid esters, or corresponding ionic emulsifiers, such as alkali metal salts of fatty alcohol sulfates, for example, sodium lauryl sulfate, sodium cetyl sulfate or sodium stearyl sulfate, which are usually used in the presence of fatty alcohols, for example cetyl stearyl alcohol or stearyl alcohol. Additives to the aqueous phase are, inter alia, agents which prevent the creams from drying out, for example polyalcohols, such as glycerol, sorbitol, propylene glycol and/or polyethylene glycols, and furthermore preservatives and odoriferous substances.

Pastes are creams and ointments having secretion-absorbing powder constituents, such as metal oxides, for example, titanium oxide or zinc oxide, and furthermore talc and/or aluminium silicates, which have the task of binding the moisture or secretions present.

Foams (i.e., liquid oil-in-water emulsions packaged in aerosol form) can be administered from pressurized containers. Propellant gases include halogenated hydrocarbons, such as polyhalogenated alkanes such as dichlorofluoromethane and dichlorotetrafluoroethane, or, preferably, non-halogenated gaseous hydrocarbons, air, N₂O, or carbon dioxide. The oily phases used are, inter alia, those mentioned above for ointments and creams, and the additives mentioned there are likewise used.

Tinctures and solutions usually comprise an aqueous-ethanolic base to which, humectants for reducing evaporation, such as polyalcohols (e.g., glycerol, glycols, polyethylene glycol) and re-oiling substances, such as fatty acid esters with lower polyethylene glycols (e.g., lipophilic substances soluble in the aqueous mixture) to substitute the fatty substances removed from the skin with the ethanol, and, if necessary or desired, other excipients and additives, are admixed.

The present invention further provides veterinary compositions comprising at least one active ingredient as above defined together with a veterinary carrier therefor. Veterinary carriers are materials for administering the composition and may be solid, liquid, or gaseous materials, which are inert or acceptable in the veterinary art and are compatible with the active ingredient. These veterinary compositions may be administered orally, parenterally, or by any other desired route.

The invention also relates to a process or method for treatment of the disease states mentioned above. The compounds can be administered prophylactically or therapeutically as such or in the form of pharmaceutical compositions, preferably in an amount, which is effective against the diseases mentioned. With a warm-blooded animal, for example, a human requiring such treatment, the compounds are used, in particular, in the form of pharmaceutical composition. A daily dose of about 0.1 to about 5 g, preferably 0.5 g to about 2 g, of a compound of the present invention is administered here for a body weight of about 70 kg.

### BRIEF DESCRIPTION OF DRAWINGS

FIG. 1 shows neuroprotective effect of novel compounds in glutamate-induced model of oxidative damage on dopaminergic neuron-like SH-SY5Y cells. The compounds of the invention at 0.1, 1 and 10 μM along with positive controls: R-lipoic acid (R-LA, 0.5, 5 and 50 μM) and deferoxamine (DFO, 1, and 100 μM) were used in co-treatment with 160 mM glutamate (Glu) for 24 hours. After 24 hours cell death was quantified by propidium iodide staining. Cell death generated by Glu was considered as 100% so the reduction of cell death was observed. All results are presented as mean±the standard error of the mean (SEM) in triplicate experiments (n=3) in three separated days. ANOVA, Tukey post hoc test; *, #P<0.05; **, ##P<0.01; ***, ###P<0.001; *P compared with vehicle with Glu 160 mM, #P compared with vehicle without Glu 160 mM. A value of P<0.05 is considered statistically significant.

FIG. 2 Glutamate-induced oxidative stress (OS) and OS-reducing activity of compounds of the invention and positive controls. The compounds of the invention at 0.1, 1 and 10 μM along with positive controls: R-lipoic acid (R-LA, 0.5, 5 and 50 μM) and deferoxamine (DFO, 1, 10 and 100 μM) were used in co-treatment with 160 mM Glu for 4 hours. After 4 hours superoxide radical formation as marker of oxidative stress was quantified by dihydroethidium staining. Oxidative stress generated by Glu was considered as 100% so the reduction of cell death was observed. All results are presented as mean±the standard error of the mean (SEM) in triplicate experiments (n=3) in three separated days. ANOVA, Tukey post hoc test; *, #P<0.05; **, ##P<0.01; ***, ###P<0.001; *P compared with vehicle with Glu 160 mM, #P compared with vehicle without Glu 160 mM. A value of P<0.05 is considered statistically significant.

### EXAMPLES OF CARRYING OUT THE INVENTION

The following examples serve to illustrate the invention without limiting the scope thereof. Unless otherwise stated, all percentages and the like amounts are based on weight. The starting materials may be obtained from commercial sources (Sigma, Aldrich, Fluka, etc.) or can be prepared as described below.

1H and 13C experiments were performed on Jeol ECX-500SS (500 MHz for 1H), and Varian^{UNITY} Inova 400 (400 MHz for 1H) instruments, using CDCl₃, DMSO-d₆, CD₃OD or THF-ds as solvents (25° C.). Chemical shifts (δ) were referenced to the residual signal of the solvent (CDCl₃, DMSO-d₆, CD₃OD or THF-d₈) and are reported in parts per million (ppm). Coupling constants (J) are reported in Hertz (Hz). NMR spectra were processed in the ACD/NMR Processor Academic Edition 12.01, MestReNova 6.0.2-5475 or JEOL Delta v5.0.5.1. HRMS analysis was performed using an LC-MS Orbitrap Elite high-resolution mass spectrometer with electrospray ionization (Dionex Ultimate 3000, Thermo Exactive plus, MA, USA). Spectra were taken at the positive and negative mode in the range of 100-1000 m/z. The samples were dissolved in MeOH and injected to the mass spectrometer over autosampler after HPLC separation: precolumn Phenomenex Gemini (C18, 50×2 mm, 2.6 μm), mobile phase isocratic MeOH/water/HCOOH 95:5:0.1. The course of the reactions was monitored by TLC on Kieselgel 60 F₂₅₄ plates (Merck) detected by spraying with 10% aqueous H₂SO₄ and heating to 400° C. Purification was performed using column chromatography on Silica gel 60 (Merck 7734).

Starting steroid derivative 1 for olefination reaction was prepared from commercially available stigmasterol (lit. Heterocycles 1982, 17, 301). All new compounds described in this invention were prepared according to the following general scheme:

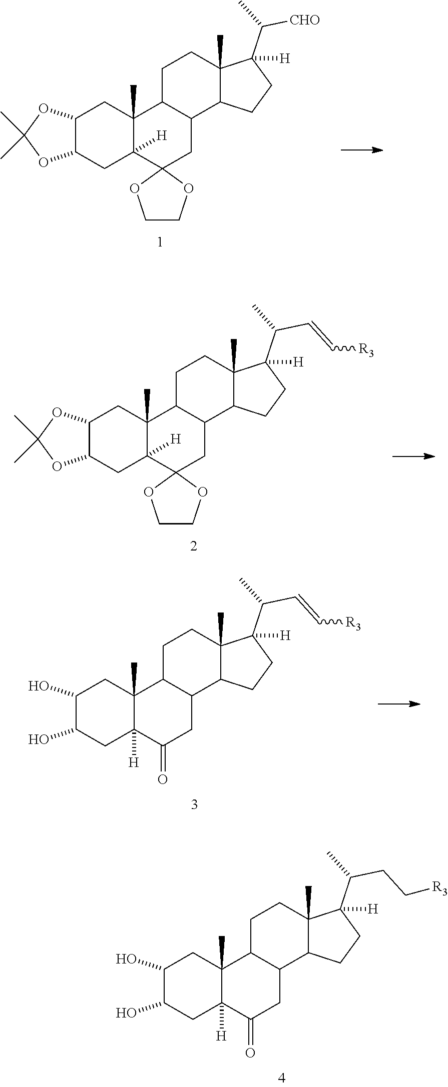

Synthesis of New Cholane and Cholestane Derivatives

### Example 1

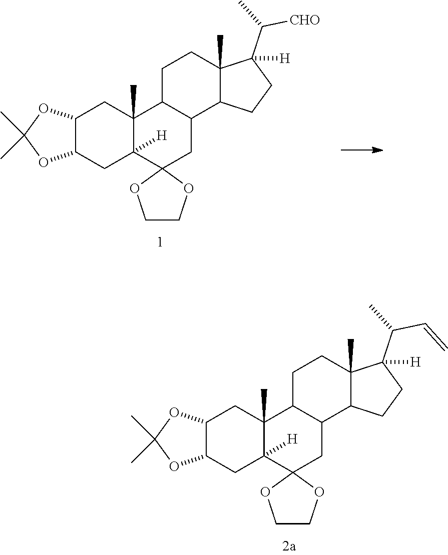

### 2α,3α-(Isopropylidenedioxy)-6,6-(ethylenedioxy)-24-nor-5α-chol-22-en (compound 2a)

To a solution of methyltriphenylphosphonium bromide (48 mg; 0.134 mmol) in anhydrous tetrahydrofuran (3 mL) was added solution of n-butyllithium in hexane (1.6 M; 85 μL; 0.134 mmol). The mixture was stirred at 0° C. for 30 minutes. Then, the solution of aldehyde 1 (30 mg; 0.067 mmol) in anhydrous tetrahydrofuran (3 mL) was added and the reaction mixture was left to reach room temperature and stirred for additional 2 hours. The reaction mixture was diluted with ethyl acetate and extracted twice with water. Organic layer was dried over calcium chloride. Solvent was evaporated under reduce pressure and crude product was subjected to silica gel chromatography (7% ethyl acetate in cyclohexane) to afford the title compound 2a.

Colourless oil, chemical formula: C₂₈H₄₄O₄, yield: 25 mg, 84%. ¹H NMR (CDCl₃, 500 MHz) δ, ppm: δ 0.69 (s, 3H, CH₃); 0.84 (s, 3H, CH₃); 1.02 (d, 3H, J=6.4 Hz, CH₃); 1.32 (s, 3H, CH₃); 1.47 (s, 3H, CH₃); 1.92 (dd, 1H, J=12.7 and 6.9 Hz); 1.97 (dt, 1H, J=12.7 and 3.2 Hz); 2.06 (m, 1H); 2.16 (m, 1H); 3.75 (m, 1H, CH₂O); 3.88-3.97 (m, 3H, CH₂O); 4.10 (m, 1H, H-2(3); 4.27 (m, 1H, H-3(3); 4.81 (dd, 1H, J=10.2 and 1.9 Hz, H-23a); 4.90 (dd, 1H, J=17.1 and 1.9 Hz, H-23b); 5.65 (ddd, 1H, J=17.1, 10.2 and 8.7 Hz, H-22). ¹³C NMR δ 12.14, 13.35, 20.04, 20.70, 21.94, 24.14, 26.55, 28.30, 28.59, 32.84, 37.98, 39.51, 41.01, 41.19, 42.43, 42.68, 45.47, 52.95, 55.37, 55.90, 64.15, 65.46, 72.82, 72.93, 107.51, 109.65, 111.54, 145.16. HRMS (API): m/z calcd for C₂₈H₄₅O₄₁[M+H]⁺ 445.3318, found 445.3319.

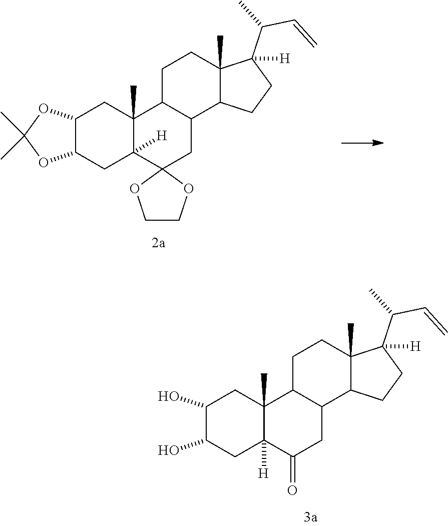

### 2α,3α-Dihydroxy-24-nor-5α-chol-22-en-6-one (compound 3a)

1M solution of hydrochloric acid (0.5 mL) was added to a solution of compound 2a (20 mg; 0.045 mmol) in tetrahydrofuran (4 mL) and the reaction mixture was heated at 40° C. for 4 hours. The reaction mixture was diluted with ethyl acetate and extracted twice with water. Organic layer was dried over calcium chloride. Solvent was evaporated under reduce pressure and crude product was subjected to silica gel chromatography (60% ethyl acetate in cyclohexane) to afford the title compound 3a.

White powder, chemical formula: C₂₃H₃₆O₃, yield: 15 mg, 93%. ¹H NMR (CDCl₃, 500 MHz) δ, ppm: δ 0.69 (s, 3H, CH₃); 0.76 (s, 3H, CH₃); 1.04 (d, 3H, J=6.7 Hz, CH₃); 1.91 (dd, 1H, J=15.0 and 3.1 Hz); 1.97-2.11 (m, 2H); 2.29 (dd, 1H, J=13.3 and 4.4 Hz); 2.68 (dd, 1H, J=12.5 and 2.8 Hz); 3.77 (m, 1H, H-2β); 4.05 (m, 1H, H-3β); 4.83 (dd, 1H, J=10.1 and 1.9 Hz, H-23a); 4.91 (dd, 1H, J=17.1 and 1.1 Hz, H-23b); 5.65 (ddd, 1H, J=17.1, 10.1 and 8.6 Hz, H-22). ¹³C NMR 612.17, 13.54, 20.04, 21.14, 23.90, 26.26, 28.15, 37.62, 39.24, 40.13, 41.11, 42.57, 42.88, 46.71, 50.67, 53.68, 55.25, 56.64, 68.24, 68.35, 111.84, 144.85, 212.23. HRMS (API): m/z calcd for C₂₃H₃₇O₃₁[M+H]⁺ 361.2743, found 361.2741.

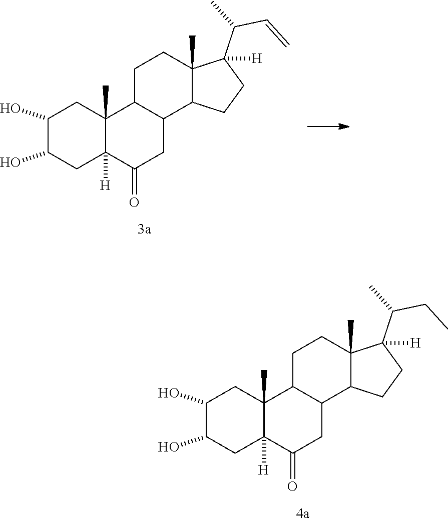

### 2α,3α-Dihydroxy-24-nor-5α-cholan-6-one (compound 4a)

Palladium on charcoal (12 mg) was added to a solution of compound 3a (12 mg; 0.033 mmol) in tetrahydrofuran (3 mL) and ethanol (1 mL). The flask with reaction mixture was evacuated and hydrogen was added from balloon. The reaction was stirred at room temperature for 18 hours. The palladium on charcoal was filtrated off, solvents were evaporated under reduce pressure and crude product was subjected to silica gel chromatography (60% ethyl acetate in cyclohexane) to afford the title compound 4a.

White powder, chemical formula: C₂₃H₃₈O₃, yield: 11 mg, 91%. ¹H NMR (CDCl₃, 500 MHz) δ, ppm: δ 0.66 (s, 3H, CH₃); 0.75 (s, 3H, CH₃); 0.83 (t, 3H, J=7.2 Hz, CH₃); 0.91 (d, 3H, J=6.4 Hz, CH₃); 1.91 (dd, 1H, J=15.2 and 3.0 Hz); 1.96-2.08 (m, 2H); 2.30 (dd, 1H, J=13.3 and 4.5 Hz); 2.68 (dd, 1H, J=12.5 and 2.7 Hz); 3.76 (m, 1H, H-2β); 4.05 (m, 1H, H-3β). ¹³C NMR δ 10.29, 11.98, 13.53, 17.98, 21.16, 23.92, 26.26, 28.17, 30.27, 36.88, 37.67, 39.35, 40.14, 42.58, 42.86, 46.77, 50.69, 53.69, 55.49, 56.62, 68.27, 68.37, 212.34. HRMS (API): m/z calcd for C₂₃H₃₉O₃ [M+H]⁺ 363.2899, found 363.2902.

### Example 2

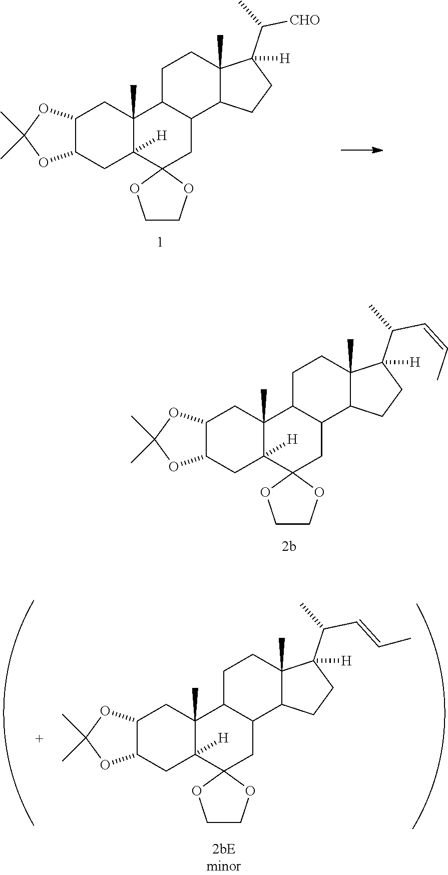

### (22Z)-2α,3α-(Isopropylidenedioxy)-6,6-(ethylenedioxy)-5α-chol-22-en (compound 2b)

To a solution of ethyltriphenylphosphonium bromide (50 mg; 0.134 mmol) in anhydrous tetrahydrofuran (3 mL) was added solution of n-butyllithium in hexane (1.6 M; 85 μL; 0.134 mmol). The mixture was stirred at 0° C. for 30 minutes. Then, the solution of aldehyde 1 (30 mg; 0.067 mmol) in anhydrous tetrahydrofuran (3 mL) was added and the reaction mixture was left to reach room temperature and stirred for additional 2 hours. The reaction mixture was diluted with ethyl acetate and extracted twice with water. Organic layer was dried over calcium chloride. Solvent was evaporated under reduce pressure and crude product was subjected to silica gel chromatography (7% ethyl acetate in cyclohexane) to afford the title compound 2b as predominantly Z isomer (less than 5% of E isomer (2bE) observed in ¹H NMR).

Colourless oil, chemical formula: C₂₉H₄₆O₄, yield: 27 mg, 87%. ¹H NMR (CDCl₃, 500 MHz) δ, ppm: δ 0.71 (s, 3H, CH₃); 0.839 (s, 3H, CH₃); 0.95 (d, 3H, J=6.7 Hz, CH₃); 1.32 (s, 3H, CH₃); 1.47 (s, 3H, CH₃); 1.60 (dd, 3H, J=6.7 and 1.5 Hz, CH₃); 1.93 (dd, 1H, J=12.7 and 6.9 Hz); 1.97 (m, 1H); 2.15 (m, 1H); 2.46 (m, 1H); 3.75 (m, 1H, CH₂O); 3.88-3.97 (m, 3H, CH₂O); 4.10 (m, 1H, H-2β); 4.27 (m, 1H, H-3β); 5.15 (m, 1H, H-22); 5.25 (m, 1H, H-23). ¹³C NMR δ 12.25, 13.10, 13.37, 20.42, 20.71, 21.95, 24.13, 26.55, 27.88, 28.60, 32.85, 33.70, 38.00, 39.55, 41.03, 42.37, 42.70, 45.48, 53.01, 55.95, 56.16, 64.15, 65.47, 72.84, 72.95, 107.51, 109.67, 120.29, 137.50. HRMS (API): m/z calcd for C₂₉H₄₇O₄ [M+H]⁺ 459.3474, found 459.3475. Selected ¹H NMR signals for 22E-isomer (2bE): δ 0.67 (s, 3H, CH₃); 0.833 (s, 3H, CH₃); 0.98 (d, 3H, J=6.7 Hz, CH₃); 5.29 (m, 1H).

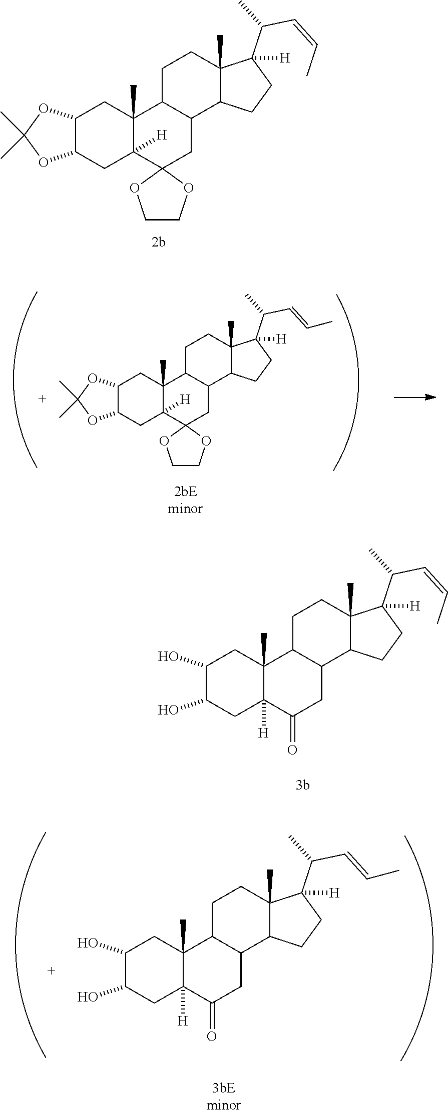

### (22Z)-2α,3α-Dihydroxy-5α-chol-22-en-6-one (compound 3b)

1M solution of hydrochloric acid (0.5 mL) was added to a solution of compound 2b (20 mg; 0.045 mmol) in tetrahydrofuran (4 mL) and the reaction mixture was heated at 40° C. for 4 hours. The reaction mixture was diluted with ethyl acetate and extracted twice with water. Organic layer was dried over calcium chloride. Solvent was evaporated under reduce pressure and crude product was subjected to silica gel chromatography (60% ethyl acetate in cyclohexane) to afford the title compound 3b (due to the presence of minor E-isomer (2bE) in the starting material 3bE (5%) can be observed in ¹H NMR).

White powder, chemical formula: C₂₄H₃₈O₃, yield: 15 mg, 92%. ¹H NMR (CDCl₃, 500 MHz) δ, ppm: δ 0.71 (s, 3H, CH₃); 0.764 (s, 3H, CH₃); 0.97 (d, 3H, J=6.7 Hz, CH₃); 1.60 (dd, 3H, J=6.7 and 1.5 Hz, CH₃); 1.92 (dd, 1H, J=15.1 and 3.2 Hz); 1.97-2.06 (m, 2H); 2.30 (dd, 1H, J=13.1 and 4.5 Hz); 2.47 (m, 1H); 2.69 (dd, 1H, J=12.5 and 2.8 Hz); 3.78 (m, 1H, H-2β); 4.05 (m, 1H, H-3β); 5.15 (m, 1H, H-22); 5.28 (m, 1H, H-23). ¹³C NMR δ12.28, 13.13, 13.55, 20.40, 21.16, 23.89, 26.26, 27.75, 33.65, 37.65, 39.27, 40.17, 42.60, 42.83, 46.74, 50.68, 53.74, 56.04, 56.68, 68.26, 68.36, 120.58, 137.20, 212.22. HRMS (API): m/z calcd for C₂₄H₃₉O_{31M}[M+H]⁺ 375.2899, found 375.2897. Selected ¹H NMR signals for 22E-isomer (3bE): δ 0.67 (s, 3H, CH₃); 0.757 (s, 3H, CH₃); 1.00 (d, 3H, J=6.7 Hz, CH₃); 5.34 (m, 1H).

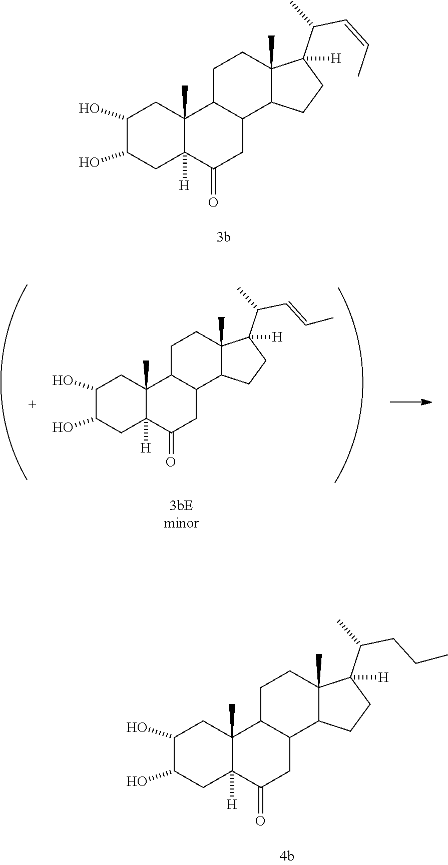

### 2α,3α-Dihydroxy-5α-cholan-6-one (compound 4b)

Palladium on charcoal (12 mg) was added to a solution of compound 3b with minor 3bE (12 mg; 0.033 mmol) in tetrahydrofuran (3 mL) and ethanol (1 mL). The flask with reaction mixture was evacuated and hydrogen was added from balloon. The reaction was stirred at room temperature for 18 hours. The palladium on charcoal was filtrated off, solvents were evaporated under reduce pressure and crude product was subjected to silica gel chromatography (60% ethyl acetate in cyclohexane) to afford the title compound 4b.

White powder, chemical formula: C₂₄H₄₀O₃, yield: 10 mg, 83%. ¹H NMR (CDCl₃, 500 MHz) δ, ppm: δ 0.66 (s, 3H, CH₃); 0.76 (s, 3H, CH₃); 0.87 (t, 3H, J=7.0 Hz, CH₃); 0.91 (d, 3H, J=6.4 Hz, CH₃); 1.92 (dd, 1H, J=15.2 and 3.3 Hz); 1.97-2.08 (m, 2H); 2.30 (dd, 1H, J=13.2 and 4.6 Hz); 2.68 (dd, 1H, J=12.6 and 2.9 Hz); 3.77 (m, 1H, H-213); 4.05 (m, 1H, H-313). ¹³C NMR δ 11.98, 13.54, 14.52, 18.56, 19.19, 21.18, 23.93, 26.26, 28.01, 35.49, 37.68, 38.20, 39.38, 40.17, 42.60, 42.92, 46.78, 50.69, 53.70, 56.05, 56.66, 68.29, 68.38, 212.26. HRMS (API): m/z calcd for C₂₄H₃₉O₂ [M−H₂O+H]⁺ 359.2950, found 359.2948.

### Example 3

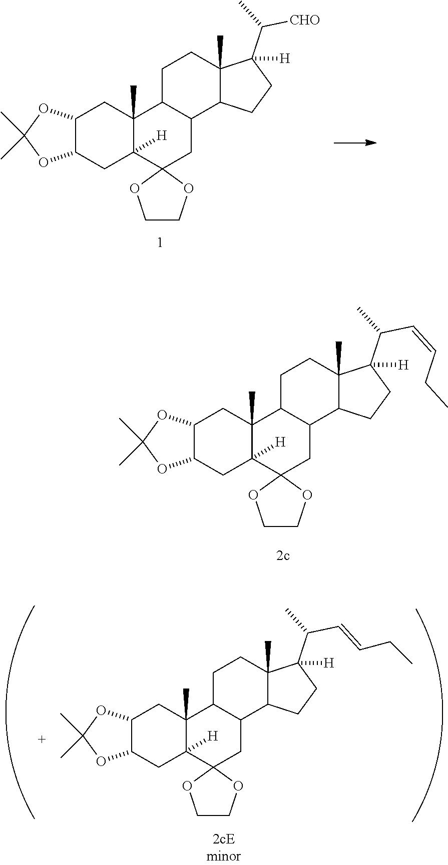

### (22Z)-2α,3α-(Isopropylidenedioxy)-6,6-(ethylenedioxy)-26,27-dinor-5α-cholest-22-en (compound 2c)

To a solution of propyltriphenylphosphonium bromide (52 mg; 0.135 mmol) in anhydrous tetrahydrofuran (3 mL) was added solution of n-butyllithium in hexane (1.6 M; 85 μL; 0.134 mmol). The mixture was stirred at 0° C. for 30 minutes. Then, the solution of aldehyde 1 (30 mg; 0.067 mmol) in anhydrous tetrahydrofuran (3 mL) was added and the reaction mixture was left to reach room temperature and stirred for additional 2 hours. The reaction mixture was diluted with ethyl acetate and extracted twice with water. Organic layer was dried over calcium chloride. Solvent was evaporated under reduce pressure and crude product was subjected to silica gel chromatography (7% ethyl acetate in cyclohexane) to afford the title compound 2c as predominantly Z isomer (7% of E isomer (2cE) observed in ¹H NMR).

Colourless oil, chemical formula: C₃₀H₄₈O₄, yield: 28 mg, 88%. ¹H NMR (CDCl₃, 500 MHz) δ, ppm: δ 0.70 (s, 3H, CH₃); 0.841 (s, 3H, CH₃); 0.95 (t, 3H, J=7.4 Hz, CH₃); 0.96 (d, 3H, J=6.7 Hz, CH₃); 1.33 (s, 3H, CH₃); 1.48 (s, 3H, CH₃); 1.91-1.99 (m, 2H); 2.00-2.09 (m, 2H); 2.16 (m, 1H); 2.42 (m, 1H); 3.75 (m, 1H, CH₂O); 3.88-3.97 (m, 3H, CH₂O); 4.11 (m, 1H, H-2β); 4.27 (m, 1H, H-3β); 5.09 (m, 1H, H-22); 5.18 (m, 1H, H-23). ¹³C NMR δ12.26, 13.38, 14.49, 20.71, 20.80, 20.86, 21.96, 24.13, 26.56, 28.06, 28.61, 32.86, 34.15, 38.00, 39.56, 41.03, 42.37, 42.70, 45.48, 53.01, 55.97, 56.02, 64.16, 65.48, 72.84, 72.95, 107.52, 109.68, 128.22, 135.93. HRMS (API): m/z calcd for C₃₀H₄₉O₄ [M+H]⁺ 473.3631, found 473.3633. Selected ¹H NMR signals for 22E-isomer (2cE): δ 0.67 (s, 3H, CH₃); 0.838 (s, 3H, CH₃); 0.99 (d, 3H, J=6.7 Hz, CH₃); 5.33 (m, 1H).

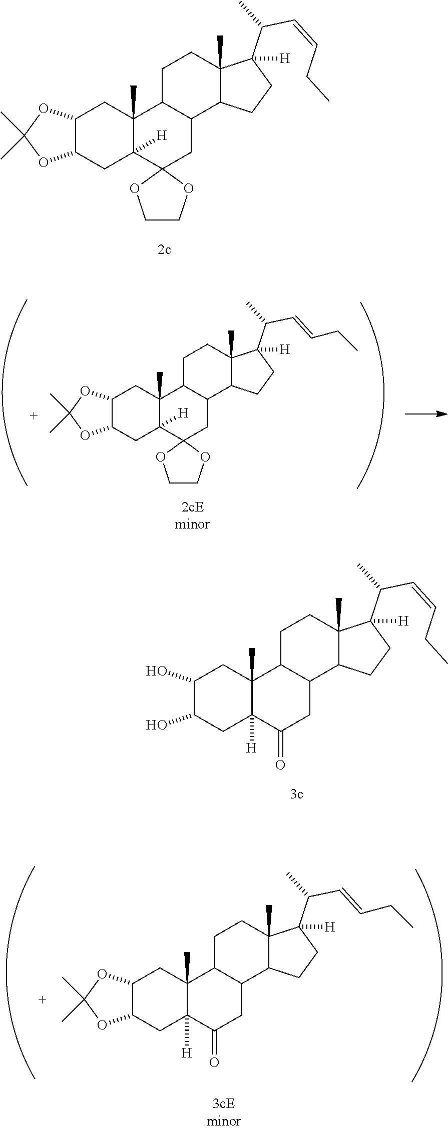

### (22Z)-2α,3α-Dihydroxy-26,27-dinor-5α-cholest-22-en-6-one (compound 3c)

1M solution of hydrochloric acid (0.5 mL) was added to a solution of compound 2c (20 mg; 0.042 mmol) in tetrahydrofuran (4 mL) and the reaction mixture was heated at 40° C. for 4 hours. The reaction mixture was diluted with ethyl acetate and extracted twice with water. Organic layer was dried over calcium chloride. Solvent was evaporated under reduce pressure and crude product was subjected to silica gel chromatography (60% ethyl acetate in cyclohexane) to afford the title compound 3c (due to the presence of minor E-isomer (2cE) in the starting material 3cE (7%) can be observed in ¹H NMR).

White powder, chemical formula: C25114.003, yield: 15 mg, 91%. ¹H NMR (CDCl₃, 500 MHz) δ, ppm: δ 0.70 (s, 3H, CH₃); 0.76 (s, 3H, CH₃); 0.95 (t, 3H, J=7.4 Hz, CH₃); 0.97 (d, 3H, J=6.7 Hz, CH₃); 1.91 (dd, 1H, J=15.3 and 3.1 Hz); 1.97-2.07 (m, 2H); 2.29 (dd, 1H, J=13.1 and 4.6 Hz); 2.43 (m, 1H); 2.68 (dd, 1H, J=12.5 and 2.8 Hz); 3.77 (m, 1H, H-2β); 4.05 (m, 1H, H-3β); 5.09 (m, 1H, H-22); 5.19 (m, 1H, H-23). ¹³C NMR δ 12.40, 13.68, 14.60, 20.89, 21.00, 21.28, 24.02, 26.40, 28.07, 34.22, 37.78, 39.41, 40.28, 42.73, 42.94, 46.87, 50.80, 53.85, 56.02, 56.82, 68.37, 68.47, 128.61, 135.74, 212.39. HRMS (API): m/z calcd for C₂₅H₄₁O₃ [M+H]⁺ 389.3056, found 389.3053. Selected ¹H NMR signals for 22E-isomer (3bE): δ 0.67 (s, 3H, CH₃); 0.74 (s, 3H, CH₃); 1.00 (d, 3H, J=6.7 Hz, CH₃); 5.35 (m, 1H).

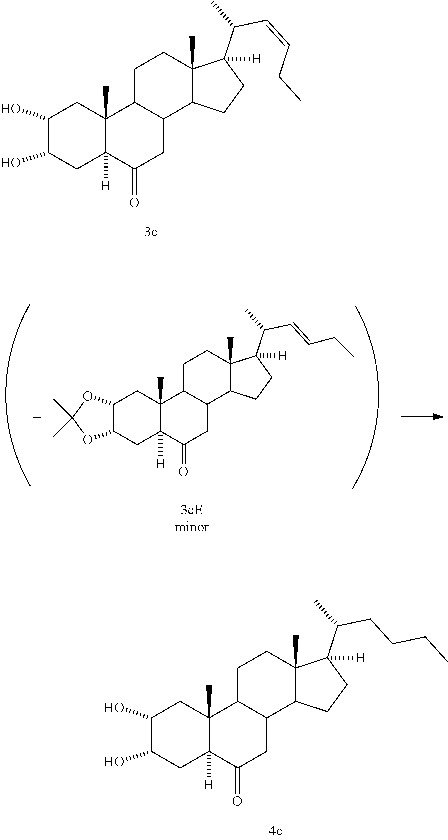

### 2α,3α-Dihydroxy-26,27-dinor-5α-cholestan-6-one (compound 4c)

Palladium on charcoal (12 mg) was added to a solution of compound 3c with minor 3cE (12 mg; 0.031 mmol) in tetrahydrofuran (3 mL) and ethanol (1 mL). The flask with reaction mixture was evacuated and hydrogen was added from balloon. The reaction was stirred at room temperature for 18 hours. The palladium on charcoal was filtrated off, solvents were evaporated under reduce pressure and crude product was subjected to silica gel chromatography (60% ethyl acetate in cyclohexane) to afford the title compound 4c.

White powder, chemical formula: C₂₅H₄₂O₃, yield: 10 mg, 83%. ¹H NMR (CDCl₃, 500 MHz) δ, ppm: δ 0.66 (s, 3H, CH₃); 0.75 (s, 3H, CH₃); 0.89 (t, 3H, J=7.0 Hz, CH₃); 0.91 (d, 3H, J=6.4 Hz, CH₃); 1.91 (dd, 1H, J=15.2 and 3.3 Hz); 1.96-2.07 (m, 2H); 2.30 (dd, 1H, J=13.2 and 4.6 Hz); 2.68 (dd, 1H, J=12.6 and 3.0 Hz); 3.76 (m, 1H, H-2β); 4.05 (m, 1H, H-3β). ¹³C NMR δ11.98, 13.53, 14.17, 18.61, 21.17, 23.11, 23.92, 26.26, 27.99, 28.28, 30.28, 35.50, 37.68, 39.37, 40.16, 42.59, 42.90, 46.78, 50.69, 53.70, 55.97, 56.65, 68.28, 68.38, 212.32. HRMS (API): m/z calcd for C₂₅H₄₃O₂ [M−H₂O+H]⁺ 391.3212, found 391.3209.

### Example 4

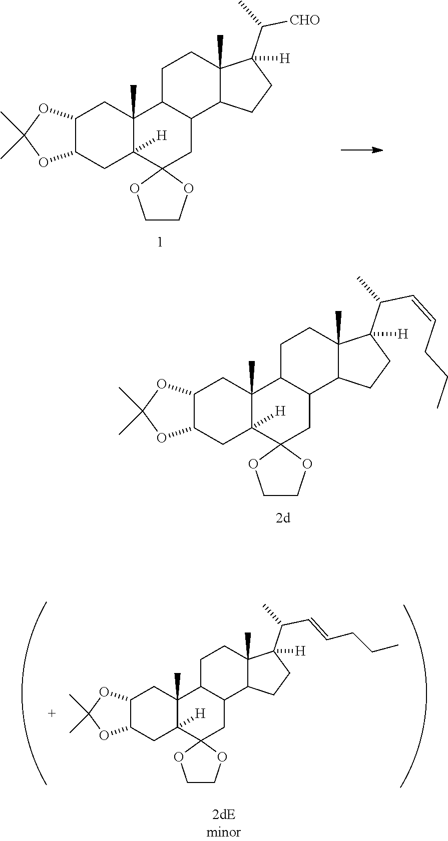

### (22Z)-2α,3α-(Isopropylidenedioxy)-6,6-(ethylenedioxy)-27-nor-5α-cholest-22-en (compound 2d)

To a solution of butyltriphenylphosphonium bromide (54 mg; 0.135 mmol) in anhydrous tetrahydrofuran (3 mL) was added solution of n-butyllithium in hexane (1.6 M; 85 μL; 0.134 mmol). The mixture was stirred at 0° C. for 30 minutes. Then, the solution of aldehyde 1 (30 mg; 0.067 mmol) in anhydrous tetrahydrofuran (3 mL) was added and the reaction mixture was left to reach room temperature and stirred for additional 2 hours. The reaction mixture was diluted with ethyl acetate and extracted twice with water. Organic layer was dried over calcium chloride. Solvent was evaporated under reduce pressure and crude product was subjected to silica gel chromatography (7% ethyl acetate in cyclohexane) to afford the title compound 2d as predominantly Z isomer (20% of E isomer (2dE) observed in ¹H NMR).

Colourless oil, chemical formula: C311-15004, yield: 27 mg, 82%. ¹H NMR (CDCl₃, 500 MHz) δ, ppm: δ 0.70 (s, 3H, CH₃); 0.84 (s, 3H, CH₃); 0.89 (t, 3H, J=7.0 Hz, CH₃); 0.91 (d, 3H, J=6.7 Hz, CH₃); 1.33 (s, 3H, CH₃); 1.48 (s, 3H, CH₃); 1.89-2.09 (m, 5H); 2.15 (m, 1H); 2.42 (m, 1H); 3.75 (m, 1H, CH₂O); 3.88-3.97 (m, 3H, CH₂O); 4.10 (m, 1H, H-2β); 4.28 (m, 1H, H-3β); 5.11-5.20 (m, 2H H-22, H-23). ¹³C NMR δ 12.28, 13.39, 13.91, 20.72, 20.74, 21.97, 22.98, 24.14, 26.57, 28.03, 28.61, 29.67, 32.86, 34.19, 38.01, 39.57, 41.03, 42.37, 42.71, 45.49, 53.02, 55.97, 56.10, 64.17, 65.48, 72.84, 72.96, 107.52, 109.68, 126.44, 136.64. HRMS (API): m/z calcd for C₃₁H₅₁O₄ [M+H]⁺ 487.3787, found 487.3783. Selected 1H NMR signals for 22E-isomer (2dE): δ 0.68 (s, 3H, CH₃); 0.84 (s, 3H, CH₃); 0.87 (t, 3H, J=7.0 Hz, CH₃); 1.00 (d, 3H, J=6.7 Hz, CH₃); 5.28 (m, 1H).

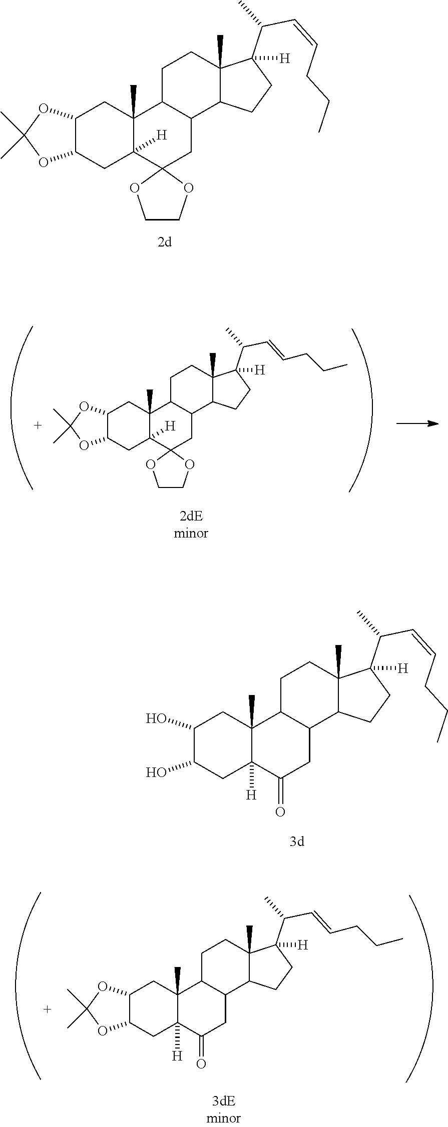

### (22Z)-2α,3α-Dihydroxy-27-nor-5α-cholest-22-en-6-one (compound 3d)

1M solution of hydrochloric acid (0.5 mL) was added to a solution of compound 2d (20 mg; 0.041 mmol) in tetrahydrofuran (4 mL) and the reaction mixture was heated at 40° C. for 4 hours. The reaction mixture was diluted with ethyl acetate and extracted twice with water. Organic layer was dried over calcium chloride. Solvent was evaporated under reduce pressure and crude product was subjected to silica gel chromatography (60% ethyl acetate in cyclohexane) to afford the title compound 3c (due to the presence of minor E-isomer (2dE) in the starting material 3dE (20%) can be observed in ¹H NMR). White powder, chemical formula: C25114.003, yield: 15 mg, 91%. ¹H NMR (CDCl₃, 500 MHz) δ, ppm: δ 0.70 (s, 3H, CH₃); 0.764 (s, 3H, CH₃); 0.91 (t, 3H, J=7.4 Hz, CH₃); 0.97 (d, 3H, J=6.7 Hz, CH₃); 1.92 (m, 1H); 1.96-2.07 (m, 4H); 2.29 (dd, 1H, J=13.1 and 4.4 Hz); 2.44 (m, 1H); 2.69 (dd, 1H, J=12.6 and 2.7 Hz); 3.78 (m, 1H, H-2β); 4.05 (m, 1H, H-3β); 5.11-5.24 (m, 2H, H-22, H-23). ¹³C NMR δ 12.29, 13.56, 13.91, 20.71, 21.16, 22.96, 23.90, 26.26, 27.91, 29.68, 34.14, 37.66, 39.28, 40.16, 42.60, 42.81, 46.74, 50.67, 53.74, 55.97, 56.69, 68.26, 68.35, 126.72, 136.33, 212.25. HRMS (API): m/z calcd for C₂₆H₄₃O₃ [M+H]⁺ 403.3212, found 403.3211. Selected ¹H NMR signals for 22E-isomer (3bE): δ 0.67 (s, 3H, CH₃); 0.758 (s, 3H, CH₃); 0.87 (t, 3H, J=7.0 Hz, CH₃); 1.01 (d, 3H, J=6.7 Hz, CH₃); 5.28 (m, 1H).

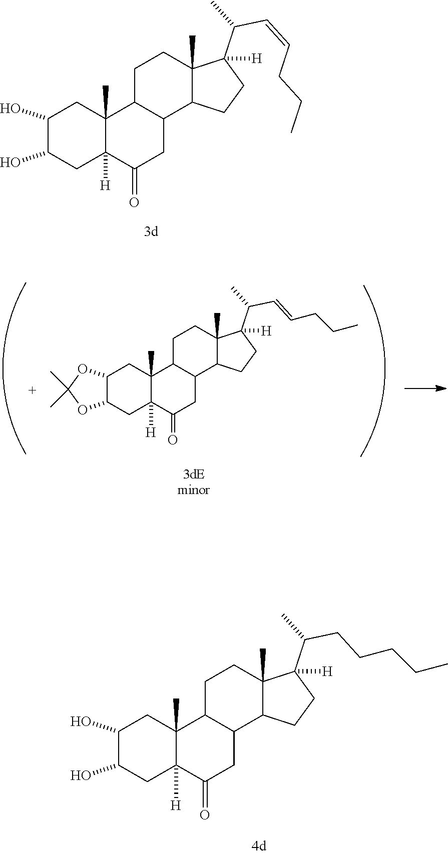

### 2α,3α-Dihydroxy-27-nor-5α-cholestan-6-one (compound 4d)

Palladium on charcoal (12 mg) was added to a solution of compound 3d with minor 3dE (12 mg; 0.030 mmol) in tetrahydrofuran (3 mL) and ethanol (1 mL). The flask with reaction mixture was evacuated and hydrogen was added from balloon. The reaction was stirred at room temperature for 18 hours. The palladium on charcoal was filtrated off, solvents were evaporated under reduce pressure and crude product was subjected to silica gel chromatography (60% ethyl acetate in cyclohexane) to afford the title compound 4d.

White powder, chemical formula: C₂₆H₄₄O₃, yield: 10 mg, 83%. ¹H NMR (CDCl₃, 500 MHz) δ, ppm: δ 0.66 (s, 3H, CH₃); 0.76 (s, 3H, CH₃); 0.89 (t, 3H, J=7.0 Hz, CH₃); 0.91 (d, 3H, J=6.4 Hz, CH₃); 1.92 (dd, 1H, J=15.2 and 3.3 Hz); 1.96-2.07 (m, 2H); 2.30 (dd, 1H, J=13.2 and 4.6 Hz); 2.68 (dd, 1H, J=12.6 and 3.0 Hz); 3.77 (m, 1H, H-2β); 4.05 (m, 1H, H-3β). ¹³C NMR δ 11.98, 13.54, 14.13, 18.61, 21.17, 22.73, 23.93, 25.70, 26.26, 27.99, 30.28, 35.64, 35.77, 37.68, 39.37, 40.16, 42.59, 42.91, 46.78, 50.69, 53.70, 55.98, 56.65, 68.29, 68.38, 212.27. HRMS (API): m/z calcd for C₂₆H₄₃O₂ [M−H₂O+H]⁺ 387.3263, found 387.3260.

TABLE 1
Examples of cholestane derivatives (Formula 1)

| | substituents | | | | | CH analysis | MS analysis |
|---|---|---|---|---|---|---|---|
| | R1 | R2 | R3 | R4 | a | [% C, % H] [calc./found] | [M + H]⁺ |
| 1 | H | OH | H | H | double | 80.18/80.16, 10.53/10.54 | 345 |
| 2 | OH | H | H | H | double | 80.18/80.16, 10.53/10.55 | 345 |
| 3 | OH | OH | H | H | double | 76.62/76.60, 10.06/10.09 | 361 |
| 4 | H | OH | H | H | single | 79.71/79.68, 11.05/11.08 | 347 |
| 5 | OH | H | H | H | single | 79.71/79.69, 11.05/11.06 | 347 |
| 6 | OH | OH | H | H | single | 76.20/76.18, 10.56/10.59 | 363 |
| 7 | H | OH | methyl | H | double | 80.39/80.38, 10.68/10.70 | 359 |
| 8 | OH | H | methyl | H | double | 80.39/80.38, 10.68/10.72 | 359 |
| 9 | OH | OH | methyl | H | double | 76.96/76.94, 10.23/10.25 | 375 |
| 10 | H | OH | H | methyl | double | 80.39/80.40, 10.68/10.70 | 359 |
| 11 | OH | H | H | methyl | double | 80.39/80.39, 10.68/10.71 | 359 |
| 12 | OH | OH | H | methyl | double | 76.96/76.93, 10.23/10.27 | 375 |
| 13 | H | OH | methyl | H | single | 79.94/79.91, 11.18/11.21 | 361 |
| 14 | OH | H | methyl | H | single | 79.94/79.93, 11.18/11.20 | 361 |
| 15 | OH | OH | methyl | H | single | 76.55/76.51, 10.71/10.74 | 377 |
| 16 | H | OH | methyl | methyl | double | 80.59/80.56, 10.82/10.85 | 373 |
| 17 | OH | H | methyl | methyl | double | 80.59/80.58, 10.82/10.83 | 373 |
| 18 | OH | OH | methyl | methyl | double | 77.27/77.25, 10.38/10.40 | 389 |
| 19 | H | OH | methyl | methyl | single | 80.16/80.14, 11.30/11.33 | 375 |
| 20 | OH | H | methyl | methyl | single | 80.16/80.13, 11.30/11.33 | 375 |
| 21 | OH | OH | methyl | methyl | single | 76.87/76.85, 10.84/10.88 | 391 |
| 22 | H | OH | ethyl | H | double | 80.59/80.57, 10.82/10.85 | 373 |
| 23 | OH | H | ethyl | H | double | 80.59/80.56, 10.82/10.86 | 373 |
| 24 | OH | OH | ethyl | H | double | 77.27/77.25, 10.38/10.40 | 389 |
| 25 | H | OH | H | ethyl | double | 80.59/80.57, 10.82/10.84 | 373 |
| 26 | OH | H | H | ethyl | double | 80.59/80.58, 10.82/10.83 | 373 |
| 27 | OH | OH | H | ethyl | double | 77.27/77.26, 10.38/10.40 | 389 |
| 28 | H | OH | ethyl | H | single | 80.16/80.14, 11.30/11.33 | 375 |
| 29 | OH | H | ethyl | H | single | 80.16/80.15, 11.30/11.34 | 375 |
| 30 | OH | OH | ethyl | H | single | 76.87/76.85, 10.84/10.86 | 391 |
| 31 | H | OH | ethyl | ethyl | double | 80.94/80.91, 11.07/11.10 | 401 |
| 32 | OH | H | ethyl | ethyl | double | 80.94/80.92, 11.07/11.08 | 401 |
| 33 | OH | OH | ethyl | ethyl | double | 77.83/77.81, 10.64/10.66 | 417 |
| 34 | H | OH | ethyl | ethyl | single | 80.54/80.53, 11.51/11.52 | 403 |
| 35 | OH | H | ethyl | ethyl | single | 80.54/80.52, 11.51/11.51 | 403 |
| 36 | OH | OH | ethyl | ethyl | single | 77.46/77.44, 11.07/11.09 | 419 |
| 37 | H | OH | methyl | ethyl | double | 80.77/80.72, 10.95/11.01 | 387 |
| 38 | OH | H | methyl | ethyl | double | 80.77/80.73, 10.95/11.00 | 387 |
| 39 | OH | OH | methyl | ethyl | double | 77.56/77.52, 10.51/10.55 | 403 |
| 40 | H | OH | ethyl | methyl | double | 80.77/80.74, 10.95/10.99 | 387 |
| 41 | OH | H | ethyl | methyl | double | 80.77/80.73, 10.95/11.01 | 387 |
| 42 | OH | OH | ethyl | methyl | double | 77.56/77.51, 10.51/10.55 | 403 |
| 43 | H | OH | ethyl | (23R)-methyl | single | 80.35/80.31, 11.41/11.46 | 389 |
| 44 | OH | H | ethyl | (23R)-methyl | single | 80.35/80.30, 11.41/11.46 | 389 |
| 45 | OH | OH | ethyl | (23R)-methyl | single | 77.18/77.15, 10.96/10.99 | 405 |
| 46 | H | OH | ethyl | (23S)-methyl | single | 80.35/80.30, 11.41/11.47 | 389 |
| 47 | OH | H | ethyl | (23S)-methyl | single | 80.35/80.30, 11.41/11.47 | 389 |
| 48 | OH | OH | ethyl | (23S)-methyl | single | 77.18/77.14, 10.96/11.01 | 405 |
| 49 | H | OH | n-propyl | H | double | 80.77/80.76, 10.95/10.98 | 387 |
| 50 | OH | H | n-propyl | H | double | 80.77/80.76, 10.95/10.99 | 387 |
| 51 | OH | OH | n-propyl | H | double | 77.56/77.54, 10.51/10.53 | 403 |
| 52 | H | OH | H | n-propyl | double | 80.77/80.75, 10.95/10.97 | 387 |
| 53 | OH | H | H | n-propyl | double | 80.77/80.76, 10.95/10.98 | 387 |
| 54 | OH | OH | H | n-propyl | double | 77.56/77.53, 10.51/10.54 | 403 |
| 55 | H | OH | n-propyl | H | single | 80.35/80.31, 11.41/11.45 | 389 |
| 56 | OH | H | n-propyl | H | single | 80.35/80.32, 11.41/11.43 | 389 |
| 57 | OH | OH | n-propyl | H | single | 77.18/77.16, 10.96/11.00 | 405 |
| 58 | H | OH | isopropyl | H | double | 80.77/80.75, 10.95/10.98 | 387 |
| 59 | OH | H | isopropyl | H | double | 80.77/80.78, 10.95/10.99 | 387 |
| 60 | OH | OH | isopropyl | H | double | 77.56/77.53, 10.51/10.53 | 403 |
| 61 | H | OH | H | isopropyl | double | 80.77/80.76, 10.95/10.98 | 387 |
| 62 | OH | H | H | isopropyl | double | 80.77/80.74, 10.95/10.99 | 387 |
| 63 | OH | OH | H | isopropyl | double | 77.56/77.53, 10.51/10.55 | 403 |
| 64 | H | OH | isopropyl | H | single | 80.35/80.33, 11.41/11.43 | 389 |
| 65 | OH | H | isopropyl | H | single | 80.35/80.33, 11.41/11.45 | 389 |
| 66 | OH | OH | isopropyl | H | single | 77.18/77.15, 10.96/10.99 | 405 |
| 67 | H | OH | n-butyl | H | double | 80.94/80.92, 11.07/11.09 | 401 |
| 68 | OH | H | n-butyl | H | double | 80.94/80.93, 11.07/11.10 | 401 |
| 69 | OH | OH | n-butyl | H | double | 77.83/77.80, 10.64/10.67 | 417 |
| 70 | H | OH | H | n-butyl | double | 80.94/80.92, 11.07/11.08 | 401 |
| 71 | OH | H | H | n-butyl | double | 80.94/80.91, 11.07/11.11 | 401 |
| 72 | OH | OH | H | n-butyl | double | 77.83/77.81, 10.64/10.66 | 417 |
| 73 | H | OH | n-butyl | H | single | 80.54/80.52, 11.51/11.54 | 403 |
| 74 | OH | H | n-butyl | H | single | 80.54/80.53, 11.51/11.52 | 403 |
| 75 | OH | OH | n-butyl | H | single | 77.46/77.44, 11.07/11.09 | 419 |
| 76 | H | OH | (R)-but-2-yl | H | double | 80.94/80.92, 11.07/11.10 | 401 |
| 77 | OH | H | (R)-but-2-yl | H | double | 80.94/80.91, 11.07/11.11 | 401 |
| 78 | OH | OH | (R)-but-2-yl | H | double | 77.83/77.82, 10.64/10.65 | 417 |
| 79 | H | OH | H | (R)-but-2-yl | double | 80.94/80.91, 11.07/11.09 | 401 |
| 80 | OH | H | H | (R)-but-2-yl | double | 80.94/80.91, 11.07/11.10 | 401 |
| 81 | OH | OH | H | (R)-but-2-yl | double | 77.83/77.81, 10.64/10.65 | 417 |
| 82 | H | OH | (R)-but-2-yl | H | single | 80.54/80.53, 11.51/11.52 | 403 |
| 83 | OH | H | (R)-but-2-yl | H | single | 80.54/80.51, 11.51/11.55 | 403 |
| 84 | OH | OH | (R)-but-2-yl | H | single | 77.46/77.45, 11.07/11.08 | 419 |
| 85 | H | OH | (S)-but-2-yl | H | double | 80.94/80.92, 11.07/11.08 | 401 |
| 86 | OH | H | (S)-but-2-yl | H | double | 80.94/80.93, 11.07/11.09 | 401 |
| 87 | OH | OH | (S)-but-2-yl | H | double | 77.83/77.80, 10.64/10.68 | 417 |
| 88 | H | OH | H | (S)-but-2-yl | double | 80.94/80.93, 11.07/11.10 | 401 |
| 89 | OH | H | H | (S)-but-2-yl | double | 80.94/80.91, 11.07/11.11 | 401 |
| 90 | OH | OH | H | (S)-but-2-yl | double | 77.83/77.81, 10.64/10.67 | 417 |
| 91 | H | OH | (S)-but-2-yl | H | single | 80.54/80.52, 11.51/11.53 | 403 |
| 92 | OH | H | (S)-but-2-yl | H | single | 80.54/80.52, 11.51/11.54 | 403 |
| 93 | OH | OH | (S)-but-2-yl | H | single | 77.46/77.45, 11.07/11.09 | 419 |
| 94 | H | OH | tert-butyl | H | double | 80.94/80.93, 11.07/11.08 | 401 |
| 95 | OH | H | tert-butyl | H | double | 80.94/80.92, 11.07/11.09 | 401 |
| 96 | OH | OH | tert-butyl | H | double | 77.83/77.81, 10.64/10.66 | 417 |
| 97 | H | OH | H | tert-butyl | double | 80.94/80.91, 11.07/11.11 | 401 |
| 98 | OH | H | H | tert-butyl | double | 80.94/80.92, 11.07/11.10 | 401 |
| 99 | OH | OH | H | tert-butyl | double | 77.83/77.82, 10.64/10.65 | 417 |
| 100 | H | OH | tert-butyl | H | single | 80.54/80.52, 11.51/11.54 | 403 |
| 101 | OH | H | tert-butyl | H | single | 80.54/80.53, 11.51/11.54 | 403 |
| 102 | OH | OH | tert-butyl | H | single | 77.46/77.45, 11.07/11.08 | 419 |
| 103 | H | OH | isobutyl | H | double | 80.94/80.92, 11.07/11.11 | 401 |
| 104 | OH | H | isobutyl | H | double | 80.94/80.91, 11.07/11.11 | 401 |
| 105 | OH | OH | isobutyl | H | double | 77.83/77.82, 10.64/10.65 | 417 |
| 106 | H | OH | H | isobutyl | double | 80.94/80.93, 11.07/11.08 | 401 |
| 107 | OH | H | H | isobutyl | double | 80.94/80.91, 11.07/11.09 | 401 |
| 108 | OH | OH | H | isobutyl | double | 77.83/77.81, 10.64/10.67 | 417 |
| 109 | H | OH | isobutyl | H | single | 80.54/80.51, 11.51/11.55 | 403 |
| 110 | OH | H | isobutyl | H | single | 80.54/80.53, 11.51/11.53 | 403 |
| 111 | OH | OH | isobutyl | H | single | 77.46/77.44, 11.07/11.10 | 419 |
| 112 | H | OH | n-pentyl | H | double | 81.10/81.07, 11.18/11.20 | 415 |
| 113 | OH | H | n-pentyl | H | double | 81.10/81.08, 11.18/11.20 | 415 |
| 114 | OH | OH | n-pentyl | H | double | 78.09/78.06, 10.77/10.79 | 431 |
| 115 | H | OH | H | n-pentyl | double | 81.10/81.06, 11.18/11.21 | 415 |
| 116 | OH | H | H | n-pentyl | double | 81.10/81.07, 11.18/11.19 | 415 |
| 117 | OH | OH | H | n-pentyl | double | 78.09/78.06, 10.77/10.80 | 431 |
| 118 | H | OH | n-pentyl | H | single | 80.71/80.68, 11.61/11.65 | 417 |
| 119 | OH | H | n-pentyl | H | single | 80.71/80.67, 11.61/11.66 | 417 |
| 120 | OH | OH | n-pentyl | H | single | 77.72/77.70, 11.18/11.21 | 433 |
| 121 | H | OH | 3-methylbut-1-yl | H | double | 81.10/81.07, 11.18/11.20 | 415 |
| 122 | OH | H | 3-methylbut-1-yl | H | double | 81.10/81.08, 11.18/11.19 | 415 |
| 123 | OH | OH | 3-methylbut-1-yl | H | double | 78.09/78.08, 10.77/10.78 | 431 |
| 124 | H | OH | H | 3-methylbut-1-yl | double | 81.10/81.08, 11.18/11.19 | 415 |
| 125 | OH | H | H | 3-methylbut-1-yl | double | 81.10/81.08, 11.18/11.20 | 415 |
| 126 | OH | OH | H | 3-methylbut-1-yl | double | 78.09/78.08, 10.77/10.78 | 431 |
| 127 | H | OH | 3-methylbut-1-yl | H | single | 80.71/80.69, 11.61/11.64 | 417 |
| 128 | OH | H | 3-methylbut-1-yl | H | single | 80.71/80.68, 11.61/11.65 | 417 |
| 129 | OH | OH | 3-methylbut-1-yl | H | single | 77.72/77.70, 11.18/11.20 | 433 |
| 130 | H | OH | (R)-2-methylbut-1-yl | H | double | 81.10/81.08, 11.18/11.20 | 415 |
| 131 | OH | H | (R)-2-methylbut-1-yl | H | double | 81.10/81.09, 11.18/11.19 | 415 |
| 132 | OH | OH | (R)-2-methylbut-1-yl | H | double | 78.09/78.07, 10.77/10.79 | 431 |
| 133 | H | OH | H | (R)-2-methylbut-1-yl | double | 81.10/81.06, 11.18/11.22 | 415 |
| 134 | OH | H | H | (R)-2-methylbut-1-yl | double | 81.10/81.07, 11.18/11.21 | 415 |
| 135 | OH | OH | H | (R)-2-methylbut-1-yl | double | 78.09/78.08, 10.77/10.78 | 431 |
| 136 | H | OH | (R)-2-methylbut-1-yl | H | single | 80.71/80.68, 11.61/11.64 | 417 |
| 137 | OH | H | (R)-2-methylbut-1-yl | H | single | 80.71/80.69, 11.61/11.63 | 417 |
| 138 | OH | OH | (R)-2-methylbut-1-yl | H | single | 77.72/77.69, 11.18/11.21 | 433 |
| 139 | H | OH | (S)-2-methylbut-1-yl | H | double | 81.10/81.08, 11.18/11.19 | 415 |
| 140 | OH | H | (S)-2-methylbut-1-yl | H | double | 81.10/81.08, 11.18/11.20 | 415 |
| 141 | OH | OH | (S)-2-methylbut-1-yl | H | double | 78.09/78.07, 10.77/10.79 | 431 |
| 142 | H | OH | H | (S)-2-methylbut-1-yl | double | 81.10/81.07, 11.18/11.21 | 415 |
| 143 | OH | H | H | (S)-2-methylbut-1-yl | double | 81.10/81.09, 11.18/11.20 | 415 |
| 144 | OH | OH | H | (S)-2-methylbut-1-yl | double | 78.09/78.08, 10.77/10.78 | 431 |
| 145 | H | OH | (S)-2-methylbut-1-yl | H | single | 80.71/80.68, 11.61/11.64 | 417 |
| 146 | OH | H | (S)-2-methylbut-1-yl | H | single | 80.71/80.68, 11.61/11.63 | 417 |
| 147 | OH | OH | (S)-2-methylbut-1-yl | H | single | 77.72/77.69, 11.18/11.22 | 433 |
| 148 | H | OH | (R)-3-methylbut-2-yl | H | double | 81.10/81.08, 11.18/11.19 | 415 |
| 149 | OH | H | (R)-3-methylbut-2-yl | H | double | 81.10/81.09, 11.18/11.19 | 415 |
| 150 | OH | OH | (R)-3-methylbut-2-yl | H | double | 78.09/78.06, 10.77/10.79 | 431 |
| 151 | H | OH | H | (R)-3-methylbut-2-yl | double | 81.10/81.08, 11.18/11.19 | 415 |
| 152 | OH | H | H | (R)-3-methylbut-2-yl | double | 81.10/81.07, 11.18/11.21 | 415 |
| 153 | OH | OH | H | (R)-3-methylbut-2-yl | double | 78.09/78.07, 10.77/10.79 | 431 |
| 154 | H | OH | (R)-3-methylbut-2-yl | H | single | 80.71/80.67, 11.61/11.65 | 417 |
| 155 | OH | H | (R)-3-methylbut-2-yl | H | single | 80.71/80.68, 11.61/11.64 | 417 |
| 156 | OH | OH | (R)-3-methylbut-2-yl | H | single | 77.72/77.70, 11.18/11.20 | 433 |
| 157 | H | OH | (S)-3-methylbut-2-yl | H | double | 81.10/81.09, 11.18/11.19 | 415 |
| 158 | OH | H | (S)-3-methylbut-2-yl | H | double | 81.10/81.09, 11.18/11.20 | 415 |
| 159 | OH | OH | (S)-3-methylbut-2-yl | H | double | 78.09/78.05, 10.77/10.80 | 431 |
| 160 | H | OH | H | (S)-3-methylbut-2-yl | double | 81.10/81.08, 11.18/11.20 | 415 |
| 161 | OH | H | H | (S)-3-methylbut-2-yl | double | 81.10/81.09, 11.18/11.21 | 415 |
| 162 | OH | OH | H | (S)-3-methylbut-2-yl | double | 78.09/78.06, 10.77/10.80 | 431 |
| 163 | H | OH | (S)-3-methylbut-2-yl | H | single | 80.71/80.69, 11.61/11.64 | 417 |
| 164 | OH | H | (S)-3-methylbut-2-yl | H | single | 80.71/80.70, 11.61/11.63 | 417 |
| 165 | OH | OH | (S)-3-methylbut-2-yl | H | single | 77.72/77.69, 11.18/11.23 | 433 |
| 166 | H | OH | (R)-pent-2-yl | H | double | 81.10/81.07, 11.18/11.20 | 415 |
| 167 | OH | H | (R)-pent-2-yl | H | double | 81.10/81.08, 11.18/11.20 | 415 |
| 168 | OH | OH | (R)-pent-2-yl | H | double | 78.09/78.07, 10.77/10.78 | 431 |
| 169 | H | OH | H | (R)-pent-2-yl | double | 81.10/81.08, 11.18/11.20 | 415 |
| 170 | OH | H | H | (R)-pent-2-yl | double | 81.10/81.07, 11.18/11.21 | 415 |
| 171 | OH | OH | H | (R)-pent-2-yl | double | 78.09/78.05, 10.77/10.80 | 431 |
| 172 | H | OH | (R)-pent-2-yl | H | single | 80.71/80.69, 11.61/11.64 | 417 |
| 173 | OH | H | (R)-pent-2-yl | H | single | 80.71/80.68, 11.61/11.64 | 417 |
| 174 | OH | OH | (R)-pent-2-yl | H | single | 77.72/77.70, 11.18/11.22 | 433 |
| 175 | H | OH | (S)-pent-2-yl | H | double | 81.10/81.06, 11.18/11.22 | 415 |
| 176 | OH | H | (S)-pent-2-yl | H | double | 81.10/81.07, 11.18/11.21 | 415 |
| 177 | OH | OH | (S)-pent-2-yl | H | double | 78.09/78.06, 10.77/10.80 | 431 |
| 178 | H | OH | H | (S)-pent-2-yl | double | 81.10/81.07, 11.18/11.21 | 415 |
| 179 | OH | H | H | (S)-pent-2-yl | double | 81.10/81.08, 11.18/11.20 | 415 |
| 180 | OH | OH | H | (S)-pent-2-yl | double | 78.09/78.07, 10.77/10.78 | 431 |
| 181 | H | OH | (S)-pent-2-yl | H | single | 80.71/80.69, 11.61/11.65 | 417 |
| 182 | OH | H | (S)-pent-2-yl | H | single | 80.71/80.67, 11.61/11.66 | 417 |
| 183 | OH | OH | (S)-pent-2-yl | H | single | 77.72/77.68, 11.18/11.23 | 433 |
| 184 | H | OH | pent-3-yl | H | double | 81.10/81.06, 11.18/11.22 | 415 |
| 185 | OH | H | pent-3-yl | H | double | 81.10/81.07, 11.18/11.21 | 415 |
| 186 | OH | OH | pent-3-yl | H | double | 78.09/78.05, 10.77/10.79 | 431 |
| 187 | H | OH | H | pent-3-yl | double | 81.10/81.08, 11.18/11.19 | 415 |
| 188 | OH | H | H | pent-3-yl | double | 81.10/81.08, 11.18/11.20 | 415 |
| 189 | OH | OH | H | pent-3-yl | double | 78.09/78.07, 10.77/10.80 | 431 |
| 190 | H | OH | pent-3-yl | H | single | 80.71/80.67, 11.61/11.66 | 417 |
| 191 | OH | H | pent-3-yl | H | single | 80.71/80.67, 11.61/11.65 | 417 |
| 192 | OH | OH | pent-3-yl | H | single | 77.72/77.70, 11.18/11.22 | 433 |
| 193 | H | OH | 1,2-ethylene | | double | 81.03/81.05, 10.34/10.38 | 371 |
| 194 | OH | H | 1,2-ethylene | | double | 81.03/81.07, 10.34/10.35 | 371 |
| 195 | OH | OH | 1,2-ethylene | | double | 77.68/77.65, 9.91/9.96 | 387 |
| 196 | H | OH | 1,2-ethylene | | single | 80.59/80.55, 10.82/10.83 | 373 |
| 197 | OH | H | 1,2-ethylene | | single | 80.59/80.54, 10.82/10.79 | 373 |
| 198 | OH | OH | 1,2-ethylene | | single | 77.27/77.30, 10.38/10.40 | 389 |
| 199 | H | OH | trimethylene | | double | 81.20/81.18, 10.48/10.50 | 385 |
| 200 | OH | H | trimethylene | | double | 81.20/81.17, 10.48/10.52 | 385 |
| 201 | OH | OH | trimethylene | | double | 77.95/77.91, 10.06/10.10 | 401 |
| 202 | H | OH | trimethylene | | single | 80.77/80.74, 10.95/10.96 | 387 |
| 203 | OH | H | trimethylene | | single | 80.77/80.75, 10.95/10.98 | 387 |
| 204 | OH | OH | trimethylene | | single | 77.56/77.52, 10.51/10.53 | 403 |
| 205 | H | OH | tetramethylene | | double | 81.35/81.31, 10.62/10.63 | 399 |
| 206 | OH | H | tetramethylene | | double | 81.35/81.33, 10.62/10.63 | 399 |
| 207 | OH | OH | tetramethylene | | double | 78.21/78.18, 10.21/10.25 | 415 |
| 208 | H | OH | tetramethylene | | single | 80.94/80.92, 11.07/11.10 | 401 |
| 209 | OH | H | tetramethylene | | single | 80.94/80.93, 11.07/11.09 | 401 |
| 210 | OH | OH | tetramethylene | | single | 77.83/77.81, 10.64/10.66 | 417 |
| 211 | H | OH | pentamethylene | | double | 81.50/81.47, 10.75/10.77 | 413 |
| 212 | OH | H | pentamethylene | | double | 81.50/81.49, 10.75/10.76 | 413 |
| 213 | OH | OH | pentamethylene | | double | 78.46/78.44, 10.35/10.38 | 429 |
| 214 | H | OH | pentamethylene | | single | 81.10/81.08, 11.18/11.20 | 415 |
| 215 | OH | H | pentamethylene | | single | 81.10/81.06, 11.18/11.22 | 415 |
| 216 | OH | OH | pentamethylene | | single | 78.09/78.05, 10.77/10.79 | 431 |
| 217 | H | OH | cyclopropyl | H | double | 81.20/81.18, 10.48/10.52 | 385 |
| 218 | OH | H | cyclopropyl | H | double | 81.20/81.17, 10.48/10.52 | 385 |
| 219 | OH | OH | cyclopropyl | H | double | 77.95/77.90, 10.06/10.09 | 401 |
| 220 | H | OH | H | cyclopropyl | double | 81.20/81.17, 10.48/10.54 | 385 |
| 221 | OH | H | H | cyclopropyl | double | 81.20/81.18, 10.48/10.54 | 385 |
| 222 | OH | OH | H | cyclopropyl | double | 77.95/77.91, 10.06/10.09 | 401 |
| 223 | H | OH | cyclopropyl | H | single | 80.77/80.74, 10.95/11.00 | 387 |
| 224 | OH | H | cyclopropyl | H | single | 80.77/80.73, 10.95/11.02 | 387 |
| 225 | OH | OH | cyclopropyl | H | single | 77.56/77.51, 10.51/10.56 | 403 |
| 226 | H | OH | cyclobutyl | H | double | 81.35/81.29, 10.62/10.66 | 399 |
| 227 | OH | H | cyclobutyl | H | double | 81.35/81.31, 10.62/10.65 | 399 |
| 228 | OH | OH | cyclobutyl | H | double | 78.21/78.17, 10.21/10.26 | 415 |
| 229 | H | OH | H | cyclobutyl | double | 81.35/81.29, 10.62/10.67 | 399 |
| 230 | OH | H | H | cyclobutyl | double | 81.35/81.28, 10.62/10.67 | 399 |
| 231 | OH | OH | H | cyclobutyl | double | 78.21/78.18, 10.21/10.24 | 415 |
| 232 | H | OH | cyclobutyl | H | single | 80.94/80.92, 11.07/11.10 | 401 |
| 233 | OH | H | cyclobutyl | H | single | 80.94/80.90, 11.07/11.11 | 401 |
| 234 | OH | OH | cyclobutyl | H | single | 77.83/77.80, 10.64/10.67 | 417 |
| 235 | H | OH | cyclopentyl | H | double | 81.50/81.45, 10.75/10.79 | 413 |
| 236 | OH | H | cyclopentyl | H | double | 81.50/81.46, 10.75/10.79 | 413 |
| 237 | OH | OH | cyclopentyl | H | double | 78.46/78.41, 10.35/10.40 | 429 |
| 238 | H | OH | H | cyclopentyl | double | 81.50/81.44, 10.75/10.80 | 413 |
| 239 | OH | H | H | cyclopentyl | double | 81.50/81.44, 10.75/10.79 | 413 |
| 240 | OH | OH | H | cyclopentyl | double | 78.46/78.43, 10.35/10.38 | 429 |
| 24 | H | OH | cyclopentyl | H | single | 81.10/81.04, 11.18/11.23 | 415 |
| 242 | OH | H | cyclopentyl | H | single | 81.10/81.06, 11.18/11.21 | 415 |
| 243 | OH | OH | cyclopentyl | H | single | 78.09/78.06, 10.77/10.81 | 431 |
| 244 | H | OH | cyclohexyl | H | double | 81.63/81.59, 10.87/10.91 | 427 |
| 245 | OH | H | cyclohexyl | H | double | 81.63/81.58, 10.87/10.92 | 427 |
| 246 | OH | OH | cyclohexyl | H | double | 78.68/78.64, 10.47/10.50 | 443 |
| 247 | H | OH | H | cyclohexyl | double | 81.63/81.59, 10.87/10.92 | 427 |
| 248 | OH | H | H | cyclohexyl | double | 81.63/81.60, 10.87/10.90 | 427 |
| 249 | OH | OH | H | cyclohexyl | double | 78.68/78.63, 10.47/10.52 | 443 |
| 250 | H | OH | cyclohexyl | H | single | 81.25/81.20, 11.29/11.34 | 429 |
| 251 | OH | H | cyclohexyl | H | single | 81.25/81.19, 11.29/11.35 | 429 |
| 252 | OH | OH | cyclohexyl | H | single | 78.33/78.28, 10.88/10.92 | 445 |

### Example 6—Viability Tests on Human Neuron-Like SH-SY5Y Cells

The SH-SY5Y human neuroblastoma cell line obtained purchased from ECACC (The European Collection of Authenticated Cell Cultures) was cultivated in Dulbecco's modified Eagle's Medium and Ham's F12 Nutrient Mixture (DMEM:F-12, 1:1), supplemented with 10% fetal bovine serum (FBS) and 1% penicillin and streptomycin at 37° C. in a humidified atmosphere 5% CO₂, 95% air in passage limit up to ECACC+20. The assay was performed in 96-well microplate with 7000 SH-SY5Y cells per well. Next day, SH-SY5Y underwent all-trans retinoic acid (ATRA)-differentiation for 48 h (ATRA 10 μM). After 48 h, old DMEM/F12 media was removed by fresh media containing tested compounds at 0.1, 1 and 10 μM concentrations for 24 h. The tested compounds were dissolved in DMSO were added into the medium. The maximum concentration of DMSO in media was kept below 0.1% (v/v). The cell viability was measured by Calcein AM (1 mg/ml ThermoFisher) viability assay. Solution of Calcein AM in PBS (0.75 μM) was pipetted to cells and incubated for 50 minutes. After that the fluorescence was measured at 488/517 nm (excitation/emission) using microplate reader Infinite M200 (TECAN). Calcein AM assay is based on the dye-intracellular-esterase cleavage of non-fluorescent dye (Calcein AM) by living cells to fluorescent dye (Calcein), while dyeing cells that lose such ability. The values in Table 4 show % of viability, relative to control of all tested compounds. The control (medium with DMSO, <0.1% (v/v)) was postulated as 100% viability (see the first line of the table). As shown in table 4, all derivatives were prove to not induce a decrease in viability.

TABLE 4
The effect of new derivatives on viability of human neuroblastoma cell
line SH-SY5Y (neuron-like phenotype) after 24 hours of treatment. The
data are expressed as percentages of DMSO control as the mean ±
SEM of two independent experiments.
Viability (% of DMSO control)

| | DMSO control | | | | | |
|---|---|---|---|---|---|---|
| | 100 | | | 0.95 | | |
| Compound | 0.1 μM | SEM± | 1 μM | SEM± | 10 μM | SEM± |
| 57 | 105.90 | 2.86 | 108.90 | 3.93 | 117.10 | 3.31 |
| 30 | 99.41 | 3.13 | 102.30 | 4.14 | 104.70 | 3.13 |
| 15 | 98.20 | 2.53 | 106.40 | 1.82 | 120.60 | 2.26 |
| 111 | 106.90 | 4.19 | 107.80 | 3.02 | 109.90 | 1.37 |
| 6 | 101.30 | 3.94 | 104.80 | 4.84 | 122.10 | 4.86 |
| | 0.5 μM | SEM± | 5 μM | SEM± | 50 μM | SEM± |
| R-LA | 96.12 | 2.68 | 96.93 | 3.59 | 101.2 | 2.09 |
| | 1 μM | SEM± | 5 μM | SEM± | 50 μM | SEM± |
| DFO | 105.7 | 8.48 | 105.5 | 8.49 | 109.5 | 5.92 |

*DMSO—dimethylsuplhoxide

### Example 17 Neuroprotective Effect of Examples on Glutamate-Induced Oxidative Damage of Human Neuron-Like SH-SYSY Cells

Similarly, to the previous test the assay was performed in 96-well microplates with 20000 cells per well. Day after the seeding of cells, SH-SYSY underwent all-trans retinoic acid (ATRA)-differentiation for 48 h (ATRA 10 μM). After 48 h, old DMEM/F12 media was removed by fresh media containing 160 mM glutamate (Glu) without or with tested compounds at 0.1, 1 and 10 μM concentrations. As positive controls R-lipoic acid (R-LA) at 0.5, 5, 50 μM and deferoxamine (DFO) at 1, 10 and 100 μM were used. After 24 h the cell death was quantified by propidium iodide staining according to literature (Stone et al. BMC Cell Biol. 2003, 4, 1) with modifications. In general, PI staining is associated with damaged cell membrane or presence of dying cells (Stone et al. BMC Cell Biol. 2003, 4, 1). Due to the loss of adherence, damaged or death cells were stained by PI solution directly in media at 1.5 μM final concentration and incubated at room temperature for 15-25 minutes at room temperature. PI stained cells were quantified at 535/617 nm (excitation/emission) by Infinite M200 Pro (Tecan) microplate reader. Cell death generated by 160 mM Glu was consider as 100% of cell death so that reduction in cell death (neuroprotective effect) was observed.

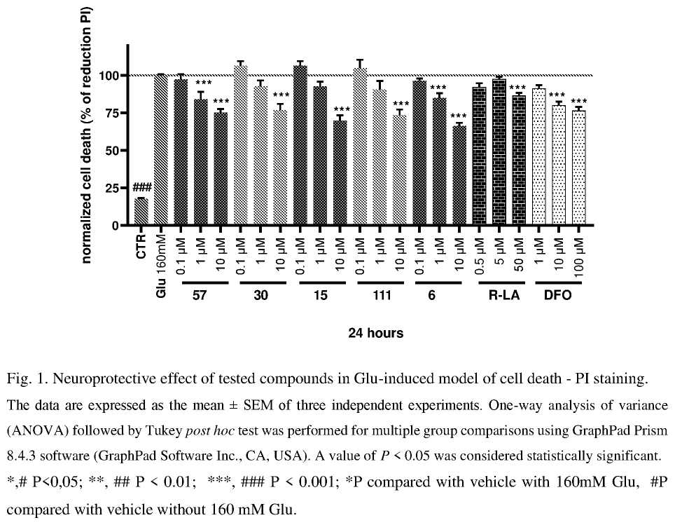

Treatment by 160 mM Glu resulted in approx. 5-times higher cell death in comparison to DMSO control. As can be seen from Table 5 and FIG. 1 all tested examples at 10 μM demonstrated comparable or higher neuroprotective effect than positive controls. Specifically, the highest effect was observed in case of compounds 15 and 6 at 10 μM with double neuroprotective effect than R-LA. Additionally, compounds showed higher efficiency than positive controls due the fact that neuroprotective effect was achieved at orders of magnitude lower concentrations (5-10 fold).

TABLE 5
Neuroprotective effects of novel compounds on human neuroblastoma
cell line SH-SY5Y (differentiated phenotype)

| Compound | Neuroprotective effect (% of control) | | | p value |
|---|---|---|---|---|
| | 0.5 μM | 5 μM | 50 μM | |
| R-LA | N/A | N/A | 13.49 | >0.001 |
| | 1 μM | 10 μM | 100 μM | |
| DFO | N/A | 19.94 | 23.55 | >0.001 |
| | 0.1 μM | 1 μM | 10 μM | |
| 57 | N/A | 15.99 | 24.88 | >0.001 |
| 30 | N/A | N/A | 23.17 | >0.001 |
| 15 | N/A | N/A | 30.08 | >0.001 |
| 111 | N/A | N/A | 26.52 | >0.001 |
| 6 | N/A | 15.00 | 33.70 | >0.001 |

N/A = no protection; The data are expressed as differences between 160 mM Glu and co-treatment groups (compounds) means, with significances after statistical analysis (ANOVA, Tukey post hoc test).

### Example 18 Measurement of Oxidative Stress on Human Neuroblastoma Cell Line SH-SYSY-Glutamate-Induced Model of PD

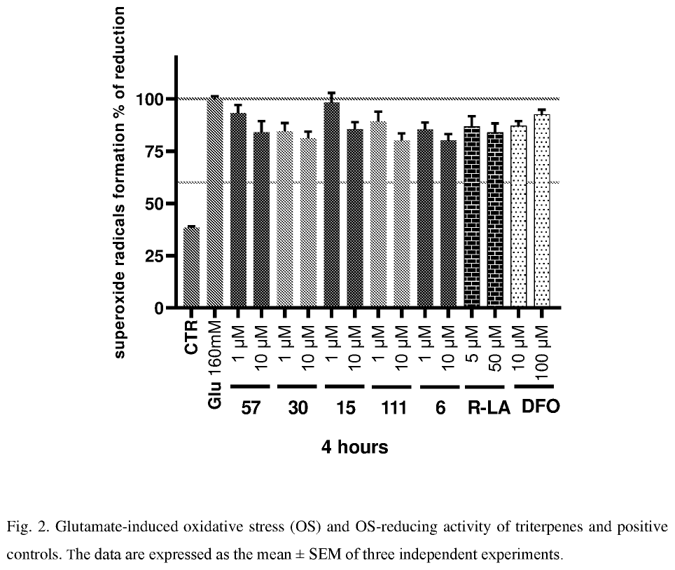

Similarly as shown in previous assay, cells grew on 96 multiwell plates at density 20000 cells per well underwent differentiation and treatment procedure for glutamate model. After 4 hour co-treatment with glutamate superoxide radical's formation as marker of oxidative stress was quantified by dihydroethidium according to Kim et al. 2017, J. Med. Food, 20, 140-151 with modifications. Briefly, neuron-like SH-SYSY cells were centrifuged at 500 g for 5 minutes and 30 seconds, then media were replaced by 10 μM DHE PBS solution and kept at room temperature for 30 minutes. After 30 minutes, DHE was read at 500 nm/580 nm (excitation/emission) by Infinite M200 Pro (Tecan) microplate reader. DHE is cell permeable dye which is selective toward superoxide radical detection. Overall oxidative stress achieved by 160 mM Glu was consider as 100% so that the reduction in oxidative stress (OS reducing effect) was observed. As shown in Table 6 and FIG. 2, 160 mM Glu induced 2.6 fold increase in superoxide radical's formation. More importantly all examples at 10 μM demonstrated comparable or slightly better OS reducing effect than antioxidant R-LA, while completely outperformed DFO. Taken together, all examples showed higher efficiency than positive controls in term of concentrations responsible for OS reducing activity (5-10 fold more effective than positive controls).

TABLE 6
Oxidative stress-induced by glutamate and OS reducing activity of
novel compounds when tested on neuron-like SH-SY5Y cells.
Superoxide radical formation (oxidative stress) % of reduction

| | average | ±SEM |
|---|---|---|
| DMSO CTR | 38.18 | 0.74 |
| Glu 160 mM | 100 | 1.52 |

| Compound | 1 μM | ±SEM | 10 μM | ±SEM |
|---|---|---|---|---|
| 57 | 93.34 | 3.83 | 83.99 | 5.33 |
| 30 | 84.54 | 3.88 | 81.08 | 3.25 |
| 15 | 98.27 | 4.65 | 85.61 | 3.28 |
| 111 | 89.17 | 4.8 | 80.21 | 3.31 |
| 6 | 85.36 | 3.38 | 80.19 | 2.95 |
| R-LA | 5 μM | ±SEM | 50 μM | ±SEM |
| | 86.72 | 5.07 | 83.91 | 4.33 |
| DFO | 10 μM | ±SEM | 100 μM | ±SEM |
| | 87.06 | 2.39 | 92.61 | 2.2 |

The data are expressed as percentages of 160 mM Glu control, the mean ± SEM of three independent experiments.

### Example 21—Formulations

The growth regulatory formulations usually contain from 0.1 to 99% by weight, especially from 0.1 to 95% by weight, of active ingredient mixture comprising a C2,C6-disubstituted-9-benzylated-9H-purine derivative of this invention, from 1 to 99.9% by weight of a solid or liquid formulation adjuvant, and from 0 to 25% by weight, especially from 0.1 to 25% by weight, of a surfactant. Whereas commercial products are usually formulated as concentrates, the end user will normally employ dilute formulations. The compositions may also comprise further ingredients, such as stabilizers, e.g., vegetable oils or epoxidized vegetable oils (epoxidized coconut, rapeseed oil or soybean oil), antifoams, e.g., silicone oil, preservatives, viscosity regulators, binders, tackifiers, and also fertilisers or other active ingredients. Preferred formulations have especially the following compositions: (%=percent by weight):

F1. Wettable powders

| | a) | b) | c) | d) |
|---|---|---|---|---|
| active ingredient mixture | 5% | 25% | 50% | 80% |
| sodium lignosulfonate | 4% | — | 3% | — |
| sodium lauryl sulphate | 2% | 3% | — | 4% |
| sodium diisobutylnaphthalene- | — | 6% | 5% | 6% |
| sulfonate | | | | |
| octylphenol polyglycol ether (7-8 mol | — | 1% | 2% | — |
| ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

F2. Suspension concentrates

| | a) | b) | c) | d) |
|---|---|---|---|---|
| active ingredient mixture | 3% | 10% | 25% | 50% |
| ethylene glycol | 5% | 5% | 5% | 5% |
| nonylphenol polyglycol ether (15 | — | 1% | 2% | — |
| mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjutants, giving a suspension concentrate from which suspensions of any desired concentration can be obtained by dilution with water.

F3. Dry Capsules

5000 capsules, each of which contain 0.25 g of one of the C2,C6-disubstituted-9-benzyl-9H-purine derivative as active ingredient, are prepared as follows:
Composition: Active ingredient: 1250 g; Talc: 180 g; Wheat starch: 120 g; Magnesium stearate: 80 g; Lactose 20 g.
Preparation process: The powdered substances mentioned are pressed through a sieve of mesh width 0.6 mm Portions of 0.33 g of the mixture are transferred to gelatine capsules with the aid of a capsule-filling machine.

F4. Soft Capsules

5000 soft gelatine capsules, each of which contain 0.05 g of one of the C2,C6-disubstituted-9-benzyl-9H-purine derivative as active ingredient, are prepared as follows:
Composition: 250 g Active ingredient+2 litres Lauroglycol
Preparation process: The powdered active ingredient is suspended in Lauroglykol® (propylene glycol laurate, Gattefossé S. A., Saint Priest, France) and ground in a wet-pulveriser to a particle size of about 1 to 3 mm Portions of in each case 0.419 g of the mixture are then transferred to soft gelatine capsules by means of a capsule-filling machine.

F5. Soft Capsules

5000 soft gelatine capsules, each of which contain 0.05 g of one of the C2,C6-disubstituted-9-benzyl-9H-purine derivative as active ingredient, are prepared as follows:
Composition: 250 g Active ingredient+1 litre PEG 400+1 litre Tween 80
Preparation process: The powdered active ingredient is suspended in PEG 400 (polyethylene glycol of Mr between 380 and about 420, Sigma, Fluka, Aldrich, USA) and Tween® 80 (polyoxyethylene sorbitan monolaurate, Atlas Chem. Inc., Inc., USA, supplied by Sigma, Fluka, Aldrich, USA) and ground in a wet-pulveriser to a particle size of about 1 to 3 mm Portions of in each case 0.43 g of the mixture are then transferred to soft gelatine capsules by means of a capsule-filling machine.

### Example 22—Formulation of Tablets with Controlled Release

One tablet contains, for example, 300-350 mg of terpenoid derivative as active ingredient. Excipient with known effect: Each tablet contains 150 to 200 mg of a retardant (Methocel, Parteck® SRP 80, Kollidon® SR, Kollidon 25, chitosan, alginate), as well as a lubricant (magnesium stearate), active substances (VH), binders (Prosolv SMCC 90).

The dosage form is a controlled release tablet.

Tablet preparation: Tablets are prepared by direct compression. First, the calculated amount of retarding component (Methocel, Parteck® SRP 80, Kollidon® SR, Kollidon 25, chitosan, alginate), weighing agent (magnesium stearate), active ingredient (VH), binder (Prosolv SMCC 90) are weighed. The resulting mixture is then homogenized in a homogenizer (Retsch MM200—Retsch GmbH, Haan). It is recommended to carry out the homogenization at three frequencies: 10 oscillations/s, 13 and 15 oscillations/s for 1 minute each. The tablet is then transferred to a hand press. The tablets are compressed at a load of 8 kN for 5 minutes. The load is selected with respect to the desired tablet strength of 0.8 to 0.8 MPa. The tablet weight is 500±5 mg.

Hydrophilic Matrix Tablets with Hypromellose

The tablets are prepared by the direct compression method as described above.

The tablet weight was 500±5 mg. Composition of hydrophilic tablets with hypromellose in wt. %:

| Formulation | A1 | A2 | A3 |
|---|---|---|---|
| Prosolv SMCC 90 | 49% | 49% | 49% |
| Methocel K4M | — | 30% | — |
| Methocel K15M | 30% | — | — |
| Methocel K100M | — | — | 30% |
| Active substance | 20% | 20% | 20% |
| Magnesium stearate | 1% | 1% | 1% |

Hydrophilic Matrix Tablets with Retarding Component Kollidon 25, Kollidon® SR, Parteck® SRP 80

The tablets are prepared by the direct compression method as described above.

The tablet weight was 500±5 mg. Composition of hydrophilic tablets in wt. %:

| Formulation | F1 | F2 | F3 | F4 | F5 |
|---|---|---|---|---|---|
| Prosolv ® SMCC 90 | 49% | 49% | 49% | 49% | 49% |
| Kollidon 25 | 30% | 20% | 10% | — | — |
| Kollidon ® SR | — | — | — | 30% | — |
| Parteck ® SRP 80 | — | — | — | — | 30% |
| Active substance | 20% | 30% | 40% | 20% | 20% |
| Magnesium stearate | 1% | 1% | 1% | 1% | 1% |

Hydrophilic Matrix Tablets Containing LubriTose™ MCC, Methocel K15M Nebo Methocel K4M

The tablets are prepared by the direct compression method as described above.

The tablet weight was 500±5 mg. Composition of hydrophilic tablets in wt. %:

| Formulation | A1 | A2 |
|---|---|---|
| LubriTose ™ MCC | 50% | 50% |
| Methocel K15M | 30% | — |
| Methocel K4M | — | 30% |
| Active substance | 20% | 20% |

What is claimed is:

1. Cholestane derivatives of the general formula I 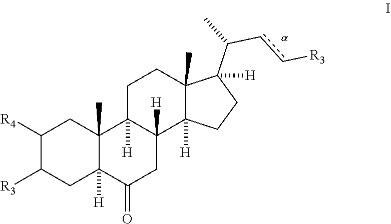
wherein,
a is single or double bond; double bond is independently selected from in either E or Z configuration;
R1, R2 is independently selected from the group consisting of hydrogen or hydroxyl group in alpha or beta configuration and their combination thereof;
R3 is independently selected from the group consisting hydrogen, linear and branched C1-5 alkyl and cycloalkyl;
R4 is independently selected from the group consisting hydrogen, methyl and ethyl;
R3, R4 is independently selected from the bivalent group consisting C2-C5 α,ω-alkylene,
and wherein the compounds 2α-hydroxy-5α-cholestan-6-one, 3α-hydroxy-5α-cholestan-6-one, 2α,3α-dihydroxy-5α-cholestan-6-one are excluded from the cholestane derivatives of general formula I,
in the form of racemates and isomers and the pharmaceutically acceptable salts thereof, in particular salts with alkali metals, ammonium or amines, or addition salts with acids.

2. Cholestane derivatives according to claim 1 with linear C1-5 alkyl in position R3, which is selected from the group consisting of methyl, ethyl, n-propyl, n-butyl, and n-pentyl.

3. Cholestane derivatives according to claim 1 with branched C1-5 alkyl in position R3, which is selected from the group consisting of isopropyl, isobutyl, sec-butyl, tert-butyl, 2-methylbutan-2-yl, 2,2-dimethylpropyl, 3-methylbutyl, pentan-2-yl, pentan-3-yl, 3-methylbutan-2-yl, and 2-methylbutyl.

4. Cholestane derivatives according to claim 1 with cycloalkyl in position R3, which is selected from the group consisting of cyclopropyl, cyclobutyl, cyclopentyl, cyclohexyl, cyclopropylmethyl, cyclobutylmethyl, cyclopentylmethyl, cyclohexylmethyl, which can be independently at each occurrence substituted by linear C1-5 alkyl.

5. Cholestane derivatives according to claim 1 with an alkylene group in position R3 and R, which are selected from the group consisting of 1,2-ethylene, trimethylene, tetramethylene, and pentamethylene.

6. Cholestane derivatives according to claim 1 with a chiral centre in position R3, which is selected from the group consisting of either R or S absolute configuration.

7. Cholestane derivatives according to claim 1, selected from the group consisting of
2α-hydroxy-24-nor-5α-cholan-6-on, 3α-hydroxy-24-nor-5α-cholan-6-one, 2α,3α-dihydroxy-24-nor-5α-cholan-6-one, 2α-hydroxy-5α-cholan-6-one, 3α-hydroxy-5α-cholan-6-one, 2α,3α-dihydroxy-5α-cholan-6-one, 2α-hydroxy-23-methyl-5α-cholan-6-one, 3α-hydroxy-23-methyl-5α-cholan-6-one, 2α,3α-dihydroxy-23-methyl-5α-cholan-6-one, 2α-hydroxy-26,27-dinor-5α-cholestan-6-one, 3α-hydroxy-26,27-dinor-5α-cholestan-6-one, 2α,3α-dihydroxy-26,27-dinor-5α-cholestan-6-one, (23R)-2α-hydroxy-23-ethyl-5α-cholan-6-one, (23R)-3α-hydroxy-23-ethyl-5α-cholan-6-one, (23R)-2α,3α-dihydroxy-23-ethyl-5α-cholan-6-one, (23S)-2α-hydroxy-23-ethyl-5α-cholan-6-one, (23S)-3α-hydroxy-23-ethyl-5α-cholan-6-one, (23S)-2α,3α-dihydroxy-23-ethyl-5α-cholan-6-one, 2α-hydroxy-27-nor-5α-cholestan-6-one, 3α-hydroxy-27-nor-5α-cholestan-6-one, 2α,3α-dihydroxy-27-nor-5α-cholestan-6-one, 2α-hydroxy-24,24-dimethyl-5α-cholan-6-one, 3α-hydroxy-24,24-dimethyl-5α-cholan-6-one, 2α,3α-dihydroxy-24,24-dimethyl-5α-cholan-6-one, 2α-hydroxy-26α-homo-27-nor-5α-cholestan-6-one, 3α-hydroxy-26α-homo-27-nor-5α-cholestan-6-one, 2α,3α-dihydroxy-26α-homo-27-nor-5α-cholestan-6-one, 2α-hydroxy-24,24,24-trimethyl-5α-cholan-6-one, 3α-hydroxy-24,24,24-trimethyl-5α-cholan-6-one, 2α,3α-dihydroxy-24,24,24-trimethyl-5α-cholan-6-one, 2α-hydroxy-27-nor-5α-campestan-6-one, 3α-hydroxy-27-nor-5α-campestan-6-one, 2α,3α-dihydroxy-27-nor-5α-campestan-6-one, 2α-hydroxy-27-nor-5α-ergostan-6-one, 3α-hydroxy-27-nor-5α-ergostan-6-one, 2α,3α-dihydroxy-27-nor-5α-ergostan-6-one; 2α-hydroxy-26α,26b-dihomo-27-nor-5α-cholestan-6-one, 3α-hydroxy-26α,26b-dihomo-27-nor-5α-cholestan-6-one, 2α,3α-dihydroxy-26α,26b-dihomo-27-nor-5α-cholestan-6-one, 2α-hydroxy-5α-campestan-6-one, 2α-hydroxy-5α-ergostan-6-one, 3α-hydroxy-5α-ergostan-6-one, 2α,3α-dihydroxy-5α-ergostan-6-one, 2α-hydroxy-24-ethyl-24,24-dimethyl-5α-cholan-6-one, 3α-hydroxy-24-ethyl-24,24-dimethyl-5α-cholan-6-one, 2α,3α-dihydroxy-24-ethyl-24,24-dimethyl-5α-cholan-6-one, 2α-hydroxy-25-methyl-5α-cholestan-6-one,3α-hydroxy-25-methyl-5α-cholestan-6-one, 2α,3α-dihydroxy-25-methyl-5α-cholestan-6-one, 2α-hydroxy-27-nor-5α-stigmastan-6-one, 3α-hydroxy-27-nor-5α-stigmastan-6-one, 2α,3α-dihydroxy-27-nor-5α-stigmastan-6-one, (24S)-2α-hydroxy-26α-homo-27-nor-5α-campestan-6-one, (24S)-3α-hydroxy-26α-homo-27-nor-5α-campestan-6-one, (24S)-2α,3α-dihydroxy-26α-homo-27-nor-5α-campestan-6-one, (24R)-2α-hydroxy-26α-homo-27-nor-5α-ergostan-6-one, (24R)-3α-hydroxy-26α-homo-27-nor-5α-ergostan-6-one, (24R)-2α,3α-dihydroxy-26α-homo-27-nor-5α-ergostan-6-one, 2α-hydroxy-22-cyclopropyl-23,24-dinor-5α-cholan-6-one, 3α-hydroxy-22-cyclopropyl-23,24-dinor-5α-cholan-6-one, 2α,3α-dihydroxy-22-cyclopropyl-23,24-dinor-5α-cholan-6-one, 2α-hydroxy-22-cyclobutyl-23,24-dinor-5α-cholan-6-one, 3α-hydroxy-22-cyclobutyl-23,24-dinor-5α-cholan-6-one, 2α,3α-dihydroxy-22-cyclobutyl-23,24-dinor-5α-cholan-6-one, 2α-hydroxy-22-cyclopentyl-23,24-dinor-5α-cholan-6-one, 3α-hydroxy-22-cyclopentyl-23,24-dinor-5α-cholan-6-one, 2α,3α-dihydroxy-22-cyclopentyl-23,24-dinor-5α-cholan-6-one, 2α-hydroxy-22-cyclohexyl-23,24-dinor-5α-cholan-6-one, 3α-hydroxy-22-cyclohexyl-23,24-dinor-5α-cholan-6-one, 2α,3α-dihydroxy-22-cyclohexyl-23,24-dinor-5α-cholan-6-one, 2α-hydroxy-23-cyclopropyl-24-nor-5α-cholan-6-one, 3α-hydroxy-23-cyclopropyl-24-nor-5α-cholan-6-one, 2α,3α-dihydroxy-23-cyclopropyl-24-nor-5α-cholan-6-one, 2α-hydroxy-23-cyclobutyl-24-nor-5α-cholan-6-one, 3α-hydroxy-23-cyclobutyl-24-nor-5α-cholan-6-one, 2α,3α-dihydroxy-23-cyclobutyl-24-nor-5α-cholan-6-one, 2α-hydroxy-23-cyclopentyl-24-nor-5α-cholan-6-one, 3α-hydroxy-23-cyclopentyl-24-nor-5α-cholan-6-one, 2α,3α-dihydroxy-23-cyclopentyl-24-nor-5α-cholan-6-one, 2α-hydroxy-23-cyclohexyl-24-nor-5α-cholan-6-one, 3α-hydroxy-23-cyclohexyl-24-nor-5α-cholan-6-one, 2α,3α-dihydroxy-23-cyclohexyl-24-nor-5α-cholan-6-one.

8. A method comprising administering a medicament comprising the cholestane derivatives of the general formula I according to claim 1.

9. A method of treatment comprising the step of administering the cholestane derivatives according to claim 1 for protection or prophylaxis of cell damage, injury and cell death.

10. A method of treatment comprising the step of administering the cholestane derivatives according to claim 1 for prophylaxis of neurodegenerative diseases.

11. The method of treatment according to claim 9, wherein the neurodegenerative disease is selected from amyotrophic lateral sclerosis, Parkinson's disease, Alzheimer's disease, Huntington's disease, progressive supranuclear palsy, corticobasal degeneration, frontotemporal dementia, Lewy body dementia, multiple system atrophy, chronic traumatic encephalopathy, spinocerebellar ataxia.

12. A pharmaceutical composition comprising one or more cholestane derivatives according to claim 1 and at least one pharmaceutically acceptable excipient.
